(19) United States
(12) Patent Application Publication
(10) Pub. No.: US 2024/0293607 A1
(43) Pub. Date: Sep. 5, 2024

(54) NEGATIVE PRESSURE WOUND THERAPY DEVICES

(71) Applicant: T.J.Smith and Nephew,Limited, Hull (GB)

(72) Inventors: Ben Alan Askem, Leeds (GB); William Davies, Edinburgh (GB); David Michael Elder, Hull (GB); James Cunningham Glencross, Musselburgh (GB); Danielle Susan Johnson, Hull (GB); Camilo Patrick Madriz, Bristol (GB); Andrea Maggiore, Hull (GB); James Donald McLusky, Edinburgh (GB); Christopher John Webb, South Ferriby (GB); Hannah Bailey Weedon, Hull (GB)

(21) Appl. No.: 18/269,935

(22) PCT Filed: Mar. 23, 2022
(86) PCT No.: PCT/EP2022/057668

§ 371 Date: Jun. 27, 2023

(30) Foreign Application Priority Data
Mar. 23, 2021 (GB) 2104021.7
Nov. 15, 2021 (GB) 2116401.7

(51) Int. Cl. A61M 1/00

(52) U.S. Cl. CPC A61M 1/98; A61M 2205/502; A61M 2205/8206

(57) ABSTRACT

A negative pressure device having a negative pressure source, a canister in fluid communication with the negative pressure source, a conduit the can couple with a wound dressing to provide negative pressure to a space beneath the wound dressing. Some arrangements of the negative pressure source can have a first noise reduction chamber and a second noise reduction chamber downstream of and in fluid communication with an outlet of a pump. The first and second noise reduction chambers can be configured to reduce noise generated by the pump and/or a level of pressure pulses in the fluid that is advanced through the negative pressure source.

### CROSS-REFERENCE TO RELATED APPLICATIONS

This application claims priority to U.K. Provisional Application No. 2104021.7, filed Mar. 23, 2021, and U.K. Provisional Application No. 2116401.7, filed Nov. 15, 2021, the entirety of each of which is hereby incorporated by reference as if fully set forth herein. The benefit of priority is claimed under the appropriate legal basis including, without limitation, under 35 U.S.C. § 119(e).

### TECHNICAL FIELD

Arrangements described herein relate to apparatuses, systems, and methods for the treatment of wounds, for example apparatuses, systems, and methods that include sources of negative pressure for use with negative pressure wound therapy dressings.

### DESCRIPTION OF THE RELATED ART

Many different types of wound dressings are known for aiding in the healing process of a human or animal. These different types of wound dressings include many different types of materials and layers, for example, gauze, pads, foam pads or multi-layer wound dressings. Topical negative pressure (TNP) therapy, sometimes referred to as vacuum assisted closure, negative pressure wound therapy, or reduced pressure wound therapy, is widely recognized as a beneficial mechanism for improving the healing rate of a wound. Such therapy is applicable to a broad range of wounds such as incisional wounds, open wounds, and abdominal wounds or the like. TNP therapy assists in the closure and healing of wounds by reducing tissue edema, encouraging blood flow, stimulating the formation of granulation tissue, removing excess exudates and may reduce bacterial load. Thus, reducing infection to the wound. Furthermore, TNP therapy permits less outside disturbance of the wound and promotes more rapid healing.

### SUMMARY OF SOME EXEMPLIFYING ARRANGEMENTS

The systems, methods and devices of this disclosure each have several innovative aspects, implementations, or aspects, no single one of which is solely responsible for the desirable attributes disclosed herein.

Disclosed herein are arrangements of a negative pressure wound therapy system that can include one or more of a pump assembly comprising a source of negative pressure configured to be fluidically connected to a wound covered by a wound dressing; a canister coupleable with the pump assembly and configured to collect fluid aspirated from a wound as a result of negative pressure being provided to the wound by the source of negative pressure; and a canister release mechanism coupled with the pump assembly and comprising an actuator coupled with one or more movable latches, the canister release mechanism being configured to cause the pump assembly to disengage the canister from the pump assembly. In some arrangements, the one or more latches can be configured to move between a first position in which the one or more latches secure the canister to the pump assembly and a second position in which the one or more latches release the canister from the pump assembly when the actuator is depressed.

Any arrangements of the negative pressure wound therapy devices, systems, and methods of using the negative pressure wound therapy devices and components of the negative pressure wound therapy devices disclosed herein can include, in additional arrangements, one or more of the following steps, features, components, and/or details, in any combination with any of the other steps, features, components, and/or details of any other arrangements disclosed herein: further including a cap coupleable with an opening on the canister; further including a filter positioned within or supported by the cap; wherein the cap comprises a shield configured to overlap at least a portion of the filter so as to inhibit exudate within the canister from splashing onto at least a portion of the filter, the shield overlapping at least 40% of a surface area of a first main surface of the filter; wherein the one or more latches are configured to engage a cap coupled with the canister in the first position and to release the cap coupled with the canister in the second position; wherein the canister release mechanism is configured to release the canister from the pump assembly with only a push of a single button; wherein the single button is supported by an external surface of a housing of the pump assembly; wherein the canister release mechanism is configured to release the canister from the pump assembly with only a single handed operation; wherein the canister release mechanism comprises a button, and wherein the actuator configured to move the one or more latches from the first position to the second position when the button is pushed; wherein the button is supported by an external surface of a housing of the device; wherein the canister release mechanism is configured to cause the canister to move away from the pump assembly when the canister release mechanism is actuated; wherein the canister release mechanism comprises at least one projection configured to push the canister away from the pump assembly when the canister release mechanism is actuated; wherein the pump assembly comprises a power cord that is electrically connected to a panel that can be removed from an outside of a housing of the pump assembly without removing or opening the housing such that the power socket and/or power cord can be replaced by replacing the panel that can be removed from an outside of a housing of the pump assembly; wherein a user interface of the pump assembly is located on an upper surface of the pump device that is oriented at an angle that is within 35° of a horizontal plane; and/or wherein the pump device has one or more tubing supports removably coupled with a housing of the pump device, wherein a tubing of the pump device extends through an enclosed opening of the one or more tubing supports and the one or more tubing support have at least one additional opening in which the tubing can be removably supported.

Also disclosed herein are arrangements of a negative pressure wound therapy system that can include one or more of a device comprising a negative pressure pump actuated by a pump motor, a battery, a display, a lower core assembly, and an upper support within a housing, a canister coupleable with the device and configured to collect fluid aspirated from a wound as a result negative pressure being provided by the negative pressure pump to a wound covered by a wound dressing and a cap coupled with an opening on the canister. In any arrangements disclosed herein, the lower core assembly can be configured to receive and support at least the pump motor and the battery. Further, in some arrangements, the upper support can be coupled with the lower core assembly, the upper support extending above the lower core assembly. Further, in some arrangements, the upper support can be configured to receive and support at least the display of the pump assembly, and the display can be removed from the pump assembly by removing the housing and by removing the upper support from the pump assembly.

Any arrangements of the negative pressure wound therapy systems, methods of using the negative pressure wound therapy systems, and components of the negative pressure wound therapy systems disclosed herein can include, in additional arrangements, one or more of the following steps, features, components, and/or details, in any combination with any of the other steps, features, components, and/or details of any other arrangements disclosed herein: further including a filter coupled with or supported by the cap; wherein the filter comprises a carbon filter; further including a hydrophobic filter coupled with or supported by the cap; wherein the cap comprises a shield configured to overlap at least a portion of the filter so as to inhibit exudate within the canister from splashing onto at least a portion of the filter, the shield overlapping at least 40%, or at least 80%, or more of a surface area of a first main surface of the filter; further including a canister release mechanism; wherein the canister release mechanism comprises one or more latches that are configured to move between a first position in which the one or more latches secure the canister to the device and a second position in which the one or more latches release the canister from the device.

Disclosed herein are arrangements of a negative pressure wound therapy device. In some arrangements, the negative pressure wound therapy device can include a negative pressure source including an inlet and an outlet, the negative pressure source being configured to provide, via a fluid flow path, negative pressure to a wound covered by a wound dressing to aspirate fluid from the wound, a first noise reduction chamber that can be positioned in the fluid flow path downstream of the negative pressure source and in fluid communication with the outlet of the negative pressure source, and a second noise reduction chamber downstream of the negative pressure source and in fluid communication with the outlet of the first noise reduction chamber. The second noise reduction chamber can be different than the first noise reduction chamber. The first noise reduction chamber can have an inlet and an outlet and can be configured to reduce noise generated by the pump and/or a level of pressure pulses in the fluid that are advanced through the pump. The second noise reduction chamber can have an inlet and an outlet, and can be configured to reduce noise generated by the pump and/or a level of pressure pulses in the fluid that is advanced through the pump and the first noise reduction chamber. The second noise reduction chamber can be spaced apart from the first noise reduction chamber.

Also disclosed herein are arrangements of a negative pressure wound therapy device. In some arrangements, the negative pressure wound therapy device can include a negative pressure source including an inlet and an outlet, the negative pressure source being configured to provide, via a fluid flow path, negative pressure to a wound covered by a wound dressing to aspirate fluid from the wound. The negative pressure wound therapy device can include a first noise reduction chamber that can be positioned in the fluid flow path downstream of the negative pressure source and in fluid communication with the outlet of the negative pressure source. The first noise reduction chamber can include an inlet and an outlet and can be configured to reduce noise generated as a result of aspirating fluid from the wound. The negative pressure wound therapy device can also include a second noise reduction chamber positioned in the fluid flow path downstream of the negative pressure source and in fluid communication with the outlet of the first noise reduction chamber. The second noise reduction chamber can include an inlet and an outlet and can be configured to reduce noise generated as a result of aspirating fluid from the wound. In some arrangements, the second noise reduction chamber can be spaced apart from the first noise reduction chamber and can be different than the first noise reduction chamber. Further, the second noise reduction chamber can be positioned to be in series with the first noise reduction chamber and to be downstream from the first noise reduction chamber. Also, in some arrangements, the second noise reduction chamber can be more proximal to an exhaust of the device than the first noise reduction chamber.

Any arrangements of the negative pressure wound therapy devices, systems, and methods of using the negative pressure wound therapy devices and components of the negative pressure wound therapy devices disclosed herein can include, in additional arrangements, one or more of the following steps, features, components, and/or details, in any combination with any of the other steps, features, components, and/or details of any other arrangements disclosed herein: wherein the negative pressure wound therapy device further includes foam positioned in at least one of the first and the second noise reduction chambers; wherein the first noise reduction chamber includes an inner wall extending across a majority of a distance between a first wall and a second wall of the first noise reduction chamber positioned adjacent to or opposite the first wall such that an opening is formed between an end of the inner wall and the second wall, the first noise reduction chamber being configured to create a passageway between the inlet and the outlet of the first noise reduction chamber that requires the fluid passing through the first noise reduction chamber to pass through the opening formed between the end of the inner wall segment and the second wall; wherein an internal volume in the first and second noise reduction chambers is greater than a volume within a first conduit in fluid communication with the inlet of at least one of the first or second noise reduction chambers and is greater than a volume within a second conduit in fluid communication with the outlet of at least one of the first or second noise reduction chambers; further including a flow module including one or more pressure sensors; further including a flow manifold including one or more pressure sensors; further including a check valve positioned in the fluid flow path and configured to prevent fluid from flowing in a reverse direction back toward the negative pressure source; wherein the first noise reduction chamber is positioned upstream of the check valve and the second noise reduction chamber is positioned downstream of the check valve; wherein the negative pressure source includes a motor, and wherein the device further includes a power source configured to power the motor; including a canister coupleable with the device and configured to collect fluid aspirated from the wound as a result negative pressure being provided by the negative pressure source to the wound and a cap coupled with an opening on the canister; further including a filter coupled with or supported by the cap; wherein the filter includes a carbon filter; and/or further including a hydrophobic filter coupled with or supported by the cap.

In some arrangements, a negative pressure wound therapy device can include a device housing and a negative pressure source supported by the device housing and configured to provide negative pressure to a wound covered by a wound dressing. The device can include a canister configured to be in fluid communication with the negative pressure source and the wound dressing. The canister can include a canister housing configured to store fluid aspirated from the wound. The canister can include a cap connected to the canister housing and configured to be connected to a device housing when the canister is removably attached to the device housing. The canister can include a fluid level sensor supported by the cap, the fluid level sensor including electrodes positioned on supports extending into an interior of the canister housing, the electrodes configured to be in fluid communication with fluid aspirated from the wound. The supports can include flanges configured to inhibit fluid from splashing onto the electrodes positioned on the supports. The fluid level sensor can be configured to detect a completed electrical circuit when the fluid aspirated from the wound comes into contact with the electrodes. The fluid level sensor can be configured to detect a canister full condition when the electrical circuit is completed. The canister can include electronic circuitry configured to communicate (for instance, wirelessly) status of the canister detected by the fluid level sensor.

Also disclosed are arrangements of methods of operating the arrangements of the devices of any of the preceding paragraphs and/or any of the systems or devices disclosed herein and arrangements of a kit including the device of any of the preceding claims and a wound dressing.

Any of the features, components, or details of any of the arrangements or arrangements disclosed in this application, including without limitation any of the apparatus arrangements and any of the negative pressure wound therapy arrangements disclosed herein, are interchangeably combinable with any other features, components, or details of any of the arrangements or arrangements disclosed herein to form new arrangements and arrangements.

### BRIEF DESCRIPTION OF THE DRAWINGS

FIG. 1A illustrates a negative pressure wound therapy system.

FIG. 1B illustrates another negative pressure wound therapy system.

FIG. 2A is an isometric view of a negative pressure wound therapy device having a pump assembly and a canister.

FIG. 2B is a front view of the negative pressure wound therapy device illustrated in FIG. 2A.

FIG. 2C is a back view of the negative pressure wound therapy device illustrated in FIG. 2A.

FIG. 2D is a side view of the negative pressure wound therapy device illustrated in FIG. 2A.

FIG. 2E is an isometric view of the back and bottom of the negative pressure wound therapy device illustrated in FIG. 2A.

FIG. 2F is a top view of the negative pressure wound therapy device illustrated in FIG. 2A.

FIG. 2G is a section view of the negative pressure wound therapy device illustrated in FIG. 2A, taken through lines 2G-2G in FIG. 2F.

FIG. 2H is an isometric view of a negative pressure wound therapy device illustrated in FIG. 2A, illustrating the canister detached from the pump assembly.

FIG. 2I illustrates a top surface of the negative pressure wound therapy device illustrated in FIG. 2A, illustrating a user interface.

FIG. 3A illustrates an exploded view of the pump assembly of the negative pressure wound therapy device illustrated in FIG. 2A.

FIG. 3B illustrates the canister of the negative pressure wound therapy device illustrated in FIG. 2A.

FIGS. 3C-3F illustrate partially exploded views of portions of the pump assembly illustrated in FIG. 3A.

FIGS. 3G-3K illustrate a portions of another arrangement of a pump assembly.

FIGS. 4A-4H illustrate a lower core assembly of the pump assembly illustrated in FIG. 3A.

FIGS. 5A-5E illustrate a cap assembly of the canister illustrated in FIG. 3B.

FIGS. 6A-6F illustrate variations of a filter that can be used with any of the negative pressure wound therapy devices disclosed herein.

FIG. 7A illustrates a tubing support of the negative pressure wound therapy device illustrated in FIG. 2A.

FIG. 7B illustrates a portion of the pump assembly illustrated in FIG. 2A.

FIGS. 8A-8D illustrate variations of a handle that can be used with any of the negative pressure wound therapy devices disclosed herein.

FIG. 9 illustrates a schematic of a control system of a negative pressure wound therapy device.

FIG. 10 illustrates another negative pressure wound therapy system.

FIGS. 11A-11D illustrate an arrangement of a canister assembly that can be used with any of the pump assembly arrangements disclosed herein.

FIGS. 12A-12D illustrate another arrangement of a canister assembly that can be used with any of the pump assembly arrangements disclosed herein.

FIGS. 13A-13B illustrate another arrangement of a canister assembly that can be used with any of the pump assembly arrangements disclosed herein.

FIGS. 14A-14D illustrate another arrangement of a canister assembly that can be used with any of the pump assembly arrangements disclosed herein.

FIG. 15A is a top, front, and left side perspective view of an arrangement of a device for applying negative pressure to a wound.

FIG. 15B is a front view of the arrangement of the device of FIG. 15A.

FIG. 15C is a back view of the arrangement of the device of FIG. 15A.

FIG. 15D is a right side view of the arrangement of the device of FIG. 15A.

FIG. 15E is a left view of the arrangement of the device of FIG. 15A.

FIG. 15F is a top view of the arrangement of the device of FIG. 15A.

FIG. 15G is a bottom view of the arrangement of the device of FIG. 15A.

FIG. 16A is a top, front, and left side perspective view of another arrangement of a device for applying negative pressure to a wound.

FIG. 16B is a front view of the arrangement of the device of FIG. 16A.

FIG. 16C is a back view of the arrangement of the device of FIG. 16A.

FIG. 16D is a right side view of the arrangement of the device of FIG. 16A.

FIG. 16E is a left view of the arrangement of the device of FIG. 16A.

FIG. 16F is a top view of the arrangement of the device of FIG. 16A.

FIG. 16G is a bottom view of the arrangement of the device of FIG. 16A.

FIG. 17A is a top, front, and left side perspective view of another arrangement of a device for applying negative pressure to a wound.

FIG. 17B is a front view of the arrangement of the device of FIG. 17A.

FIG. 17C is a back view of the arrangement of the device of FIG. 17A.

FIG. 17D is a right side view of the arrangement of the device of FIG. 17A.

FIG. 17E is a left view of the arrangement of the device of FIG. 17A.

FIG. 17F is a top view of the arrangement of the device of FIG. 17A.

FIG. 17G is a bottom view of the arrangement of the device of FIG. 17A.

FIG. 18A is a top, front, and left side perspective view of another arrangement of a device for applying negative pressure to a wound.

FIG. 18B is a front view of the arrangement of the device of FIG. 18A.

FIG. 18C is a back view of the arrangement of the device of FIG. 18A.

FIG. 18D is a right side view of the arrangement of the device of FIG. 18A.

FIG. 18E is a left view of the arrangement of the device of FIG. 18A.

FIG. 18F is a top view of the arrangement of the device of FIG. 18A.

FIG. 18G is a bottom view of the arrangement of the device of FIG. 18A.

### DETAILED DESCRIPTION

Arrangements disclosed herein relate to systems and methods of treating and/or monitoring a wound. Some arrangements of the negative pressure wound therapy devices disclosed herein can include a negative pressure source configured to be connected and/or fluidically coupled, via a fluid flow path, to a wound covered by a wound dressing and provide negative pressure to a wound.

Throughout this specification reference is made to a wound. The term wound is to be broadly construed and encompasses open and closed wounds in which skin is torn, cut or punctured or where trauma causes a contusion, or any other superficial or other conditions or imperfections on the skin of a patient or otherwise that benefit from pressure treatment. A wound is thus broadly defined as any damaged region of tissue where fluid may or may not be produced. Examples of such wounds include, but are not limited to, abdominal wounds or other large or incisional wounds, abdominal wounds with open viscera, abdominal compartment syndrome, burns, partial thickness burns, either as a result of surgery, trauma, sterniotomies, fasciotomies, or other conditions, dehisced wounds, acute wounds, sub-acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

Arrangements of systems and methods disclosed herein can be used with topical negative pressure (“TNP”) or reduced pressure therapy systems. Briefly, negative pressure wound therapy assists in the closure and healing of many forms of “hard to heal” wounds by reducing tissue oedema, encouraging blood flow and granular tissue formation, or removing excess exudate and can reduce bacterial load (and thus infection risk). In addition, the therapy allows for less disturbance of a wound leading to more rapid healing. TNP therapy systems can also assist in the healing of surgically closed wounds by removing fluid. TNP therapy can help to stabilize the tissue in the apposed position of closure. A further beneficial use of TNP therapy can be found in grafts and flaps where removal of excess fluid is important and close proximity of the graft to tissue is required in order to ensure tissue viability.

As used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects pressure that is X mmHg below 760 mmHg or, in other words, a pressure of (760-X) mmHg. In addition, negative pressure that is “less” or “smaller” than X mmHg corresponds to pressure that is closer to atmospheric pressure (for example, −40 mmHg is less than-60 mmHg). Negative pressure that is “more” or “greater” than-X mmHg corresponds to pressure that is further from atmospheric pressure (for example, −80 mmHg is more than-60 mmHg). In some cases, local ambient atmospheric pressure is used as a reference point, and such local atmospheric pressure may not necessarily be, for example, 760 mmHg.

Systems and methods disclosed herein can be used with other types of treatment in addition to or instead of reduced pressure therapy, such as irrigation, removal of irrigation fluids, ultrasound, heat or cold, neuro stimulation, or the like. In some cases, disclosed systems and methods can be used for wound monitoring without application of additional therapy. Systems and methods disclosed herein can be used in conjunction with a dressing, including with compression dressing, reduced pressure dressing, or the like.

A healthcare provider, such as a clinician, nurse, or the like, can provide a TNP prescription specifying, for example, the pressure level or time of application. However, the healing process is different for each patient and the prescription may affect the healing process in a way the clinician or healthcare provider did not expect at the time of devising the prescription. A healthcare provider may try to adjust the prescription as the wound heals (or does not heal), but such process may require various appointments that can be time consuming and repetitive. Arrangements disclosed herein provide systems, devices, or methods of efficiently adjusting TNP prescriptions and delivering effective TNP therapy.

### Wound Therapy System

Any implementations of the negative pressure wound therapy devices or systems disclosed herein can have a pump assembly (also referred to herein as a device or a pump device) having a core assembly. Some arrangements of the core assembly can include all or mostly all of the electrical and mechanical components and features required for the user interface, negative pressure control, and battery operation. The core assembly can include a printed circuit board assembly (PCBA), which can be an electronic assembly that can include two system microcontrollers. The PCBA can include a main controller, which can control the operation of one or more of the user interface, communications interfaces, and alarm generation. The PCBA can include a motor controller (sometimes referred to a pump controller), which can one or more of control the operation of the pump or monitor other signals, such as temperature, pump voltage/current, or the like. Any implementations of the system disclosed herein can have two microcontrollers to provide some redundancy to the system. In this arrangement, if one controller fails, then the other can respond and ensure that the system is fail safe. The two microcontrollers can share a regular communication to check that the other is still functioning. In any arrangements, the core assembly can include a display, which can be one or more of a color display or a touch screen.

Any implementations of the core assembly can include a flow module (or flow manifold). In some arrangements the flow module can include a two-part housing that forms internal pathways when assembled. The flow module can be configured to allow the canister connection, pressure sensors, self-test solenoid and check valve to be integrated with minimal external tubing. This reduces the chances of any internal tubing becoming trapped or kinked. The flow module can have one or two or more pressure sensors. The flow module can have two pressure sensors each situated on each side of the solenoid of the flow module. The pressure sensors can allow for air flow measurement by measuring the pressure drop across the solenoid. A check valve can be used to stop any back-leakage due to wear on the motor vales

In some arrangements, plastic moldings are used for the core module or core assembly structure. Each module within the pump assembly can be supported by a separate plastic molding so that each module can be entirely supported on a separate plastic molding.

The negative pressure wound treatment system 100 can have a canister locking mechanism between the pump assembly 160 and the canister 162. In some arrangements, the locking mechanism can have a sprung mechanism that can selectively engage or secure to the canister cap, which can be threadedly or otherwise coupled with the canister (e.g., welded, glued, etc.). A canister release button can be used to unlock the locking mechanism and allow the canister to be removed. Some arrangements of the canister locking/unlocking mechanism can be configured to be operated with a single hand, to assist users who may only have one hand or many only have one strong hand to remove the canister from the pump assembly. For example and without limitation, the canister locking mechanism can be unlocked so that the canister can be removed from the pump assembly by pushing the canister release button. The negative pressure wound treatment system 100 can be configured to unlock the canister locking mechanism and, in some arrangements, to cause the pump assembly to exert a force on the canister to cause the canister to physically separate from the pump assembly. In other arrangements, the negative pressure wound treatment system 100 can be configured to enable a user to remove the canister from the pump assembly using only one hand (i.e., in a single handed operation). Again, some arrangements of the negative pressure wound treatment system 100 can be configured such that the canister can be relatively easily separated from the pump assembly by pushing the canister release button with one hand or one finger. In some arrangements, the use of this locking mechanism, while making the pump unit slightly more complex, is beneficial since the locking mechanism allows for a simpler canister design (no separate clips) and gives a good user experience. A lower core assembly or support can provide support for the battery, pump and flow module. A core upper component can support the PCBA and display.

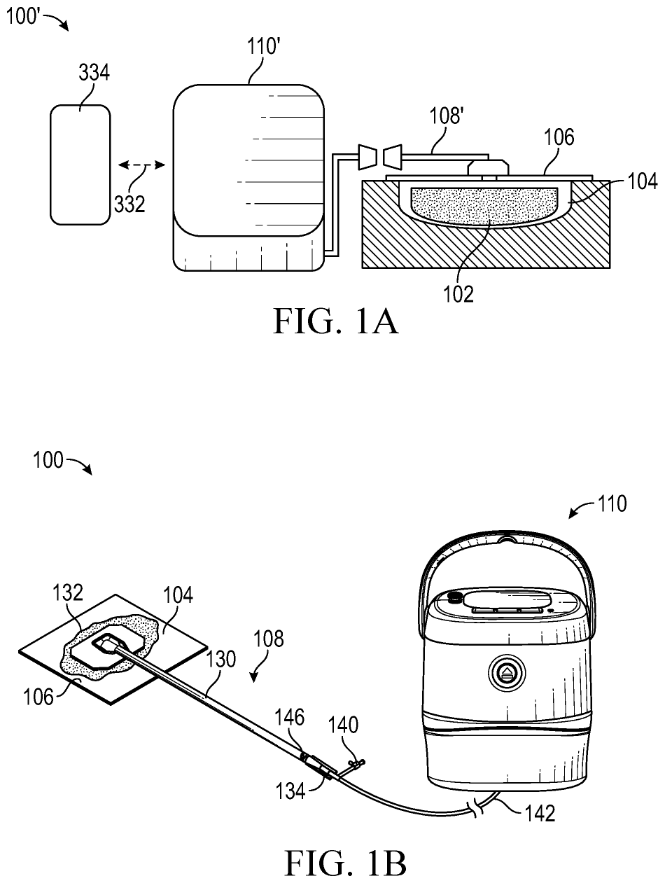

FIG. 1A schematically illustrates a negative pressure wound treatment system 100 (sometimes referred to as a reduced or negative pressure wound therapy system, a TNP system, or a wound treatment system). In any implementations disclosed herein, though not required, the negative pressure wound treatment system 100 can include a wound filler 102 placed on or inside a wound 104 (which may be a cavity). The wound 104 can be sealed by a wound cover 106, which can be a drape, such that the wound cover 106 can be in fluidic communication with the wound 104. The wound filler 102 in combination with the wound cover 106 can be referred to as a wound dressing. A tube or conduit 108 (also referred to herein as a flexible suction adapter or a fluidic connector) can be used to connect the wound cover 106 with a wound therapy device 110 (sometimes as a whole or partially referred to as a “pump assembly”) configured to supply reduced or negative pressure. The conduit 108 can be a single or multi-lumen tube. A connector 112 can be used to removably and selectively couple a conduit or tube 142 with the conduit 108.

In any of the systems disclosed herein, a wound therapy device can be canisterless, wherein, for example and without limitation, wound exudate is collected in the wound dressing or is transferred via a conduit for collection at another location. However, any of the wound therapy devices disclosed herein can include or support a canister.

Additionally, with any of the wound therapy systems disclosed herein, any of the wound therapy devices can be mounted to or supported by the wound dressing or adjacent to the wound dressing. The wound filler 102 can be any suitable type, such as hydrophilic or hydrophobic foam, gauze, inflatable bag, and so on. The wound filler 102 can be conformable to the wound 104 such that the wound filler 102 substantially fills the cavity of the wound 104. The wound cover 106 can provide a substantially fluid impermeable seal over the wound 104. The wound cover 106 can have a top side and a bottom side. The bottom side can adhesively (or in any other suitable manner) seal with the wound 104, for example by sealing with the skin around the wound 104. The conduit 108 or any other conduit disclosed herein can be formed from polyurethane, PVC, nylon, polyethylene, silicone, or any other suitable material.

The wound cover 106 can have a port (not shown) configured to receive an end of the conduit 108. In some cases, the conduit 108 can otherwise pass through or under the wound cover 106 to supply reduced pressure to the wound 104 so as to maintain a desired level of reduced pressure in the wound 104. The conduit 108 can be any suitable article configured to provide at least a substantially sealed fluid flow pathway or path between the wound therapy device 110 and the wound cover 106, so as to supply the reduced pressure provided by the wound therapy device 110 to wound 104.

The wound cover 106 and the wound filler 102 can be provided as a single article or an integrated single unit. In some cases, no wound filler is provided and the wound cover by itself may be considered the wound dressing. The wound dressing can then be connected, via the conduit 108, to a source of negative pressure of the wound therapy device 110. In some cases, though not required, the wound therapy device 110 can be miniaturized and portable, although larger conventional negative pressure sources (or pumps) can also be used.

The wound cover 106 can be located over a wound site to be treated. The wound cover 106 can form a substantially sealed cavity or enclosure over the wound. The wound cover 106 can have a film having a high water vapour permeability to enable the evaporation of surplus fluid, and can have a superabsorbing material contained therein to safely absorb wound exudate. In some cases, the components of the TNP systems described herein can be particularly suited for incisional wounds that exude a small amount of wound exudate.

The wound therapy device 110 can operate with or without the use of an exudate canister. In some cases, as is illustrated, the wound therapy device 110 can include an exudate canister. In some cases, configuring the wound therapy device 110 and conduit 108 so that the conduit 108 can be quickly and easily removed from the wound therapy device 110 can facilitate or improve the process of wound dressing or pump changes, if necessary. Any of the pump assemblies disclosed herein can have any suitable connection between the conduit 108 and the pump.

The wound therapy device 110 can deliver negative pressure of approximately −80 mmHg, or between about −20 mmHg and −200 mmHg. Note that these pressures are relative to normal ambient atmospheric pressure thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also in some cases a pressure range of below −75 mmHg can be used. Alternatively, a pressure range of over approximately −100 mmHg, or even −150 mmHg, can be supplied by the wound therapy device 110.

As will be described in greater detail below, the negative pressure wound treatment system 100 can be configured to provide a connection 332 to a separate or remote computing device 334′. The connection 332′ can be wired or wireless (such as, Bluetooth, Bluetooth low energy (BLE), Near-Field Communication (NFC), WiFi, or cellular). The remote computing device 334′ can be a smartphone, a tablet, a laptop or another standalone computer, a server (such as, a cloud server), another pump device, or the like.

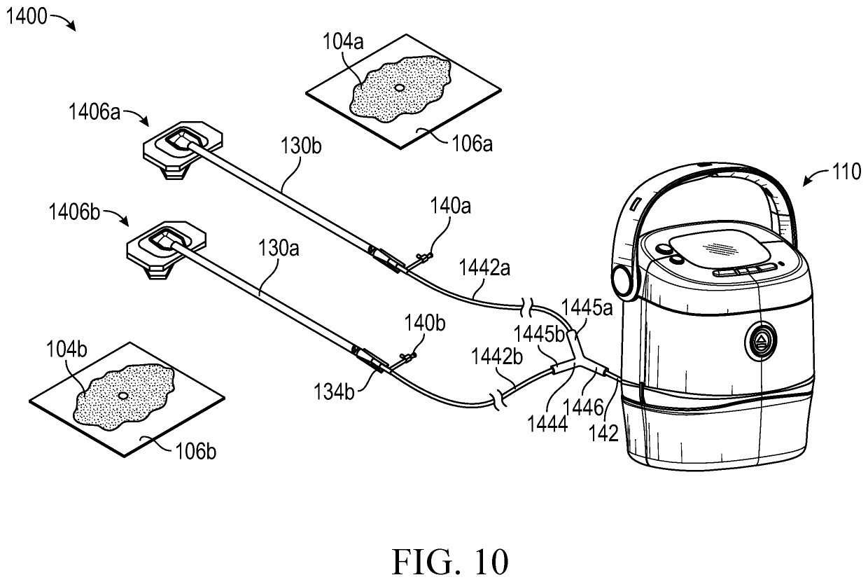

FIG. 1B illustrates another negative pressure wound treatment system 100. The negative pressure wound treatment system 100 can have any of the components, features, or other details of any of the other negative pressure wound treatment system disclosed herein, including without limitation the negative pressure wound treatment system 100′ illustrated in FIG. 1A or the negative pressure wound treatment system 1400 illustrated in FIG. 10, in combination with or in place of any of the components, features, or other details of the negative pressure wound treatment system 100 shown in FIG. 1B and/or described herein. The negative pressure wound treatment system 100 can have a wound cover 106 over a wound 104 that can seal the wound 104. A conduit 108, such as a single or multi lumen tube can be used to connect the wound cover 106 with a wound therapy device 110 (sometimes as a whole or partially referred to as a “pump assembly”) configured to supply reduced or negative pressure. The wound cover 106 can be in fluidic communication with the wound 104.

With reference to FIG. 1B, the conduit 108 can have a bridge portion 130 that can have a proximal end portion and a distal end portion (the distal end portion being closer to the wound 104 than the proximal end portion, and an applicator 132 at the distal end of the bridge portion 130 forming the flexible suction adapter (or conduit) 108. A connector 134 can be disposed at the proximal end of the bridge portion 130, so as to connect to at least one of the channels that can extend along a length of the bridge portion 130 of the conduit 108 shown in FIG. 1B. A cap 140 can be coupled with a portion of the conduit 108 and can, in some cases, as illustrated, be attached to the connector 134. The cap 140 can be useful in preventing fluids from leaking out of the proximal end of the bridge portion 130. The conduit 108 can be a Soft Port manufactured by Smith & Nephew. As mentioned, the negative pressure wound treatment system 100 can include a source of negative pressure, such as the device 110, capable of supplying negative pressure to the wound 104 through the conduit 108. Though not required, the device 110 can also include a canister or other container for the storage of wound exudates and other fluids that can be removed from the wound.

The device 110 can be connected to the connector 134 via a conduit or tube 142. In use, the applicator 132 can be placed over an aperture formed in a cover 106 that is placed over a suitably-prepared wound or wound 104. Subsequently, with the wound therapy device 110 connected via the tube 142 to the connector 134, the wound therapy device 110 can be activated to supply negative pressure to the wound. Application of negative pressure can be applied until a desired level of healing of the wound is achieved.

The bridge portion 130 can comprise an upper channel material or layer positioned between an upper layer and an intermediate layer, with a lower channel material or layer positioned between the intermediate layer and a bottom layer. The upper, intermediate, and lower layers can have elongate portions extending between proximal and distal ends and can include a material that is fluid-impermeable, for example polymers such as polyurethane. It will of course be appreciated that the upper, intermediate, and lower layers can each be constructed from different materials, including semi-permeable materials. In some cases, one or more of the upper, intermediate, and lower layers can be at least partially transparent. In some instances, the upper and lower layers can be curved, rounded or outwardly convex over a majority of their lengths.

The upper and lower channel layers can be elongate layers extending from the proximal end to the distal end of the bridge 130 and can each preferably comprise a porous material, including for example open-celled foams such as polyethylene or polyurethane. In some cases, one or more of the upper and lower channel layers can be comprised of a fabric, for example a knitted or woven spacer fabric (such as a knitted polyester 3D fabric, Baltex 7970®, or Gehring 879®) or a nonwoven material, or terry-woven or loop-pile materials. The fibers may not necessarily be woven, and can include felted and flocked (including materials such as Flotex®) fibrous materials. The materials selected are preferably suited to channeling wound exudate away from the wound and for transmitting negative pressure or vented air to the wound site, and can also confer a degree of kinking or occlusion resistance to the channel layers. In one example, the upper channel layer can include an open-celled foam such as polyurethane, and the lower channel layer can include a fabric. In another example, the upper channel layer is optional, and the system can instead be provided with an open upper channel. The upper channel layer can have a curved, rounded or upwardly convex upper surface and a substantially flat lower surface, and the lower channel layer can have a curved, rounded or downwardly convex lower surface and a substantially flat upper surface.

The fabric or material of any components of the bridge 130 can have a three-dimensional (3D) structure, where one or more types of fibers form a structure where the fibers extend in all three dimensions. Such a fabric can in some cases aid in wicking, transporting fluid or transmitting negative pressure. In some cases, the fabric or materials of the channels can include several layers of material stacked or layered over each other, which can in some cases be useful in preventing the channel from collapsing under the application of negative pressure. The materials used in some implementations of the conduit 108 can be conformable and pliable, which can, in some cases, help to avoid pressure ulcers and other complications which can result from a wound treatment system being pressed against the skin of a patient.

The distal ends of the upper, intermediate, and lower layers and the channel layers can be enlarged at their distal ends (to be placed over a wound site), and can form a “teardrop” or other enlarged shape. The distal ends of at least the upper, intermediate, and lower layers and the channel layers can also be provided with at least one through aperture. This aperture can be useful not only for the drainage of wound exudate and for applying negative pressure to the wound, but also during manufacturing of the device, as these apertures can be used to align these respective layers appropriately.

In some implementations, a controlled gas leak 146 (sometimes referred to as gas leak, air leak, or controlled air leak) can be disposed on the bridge portion 130, for example at the proximal end thereof. This air leak 146 can comprise an opening or channel extending through the upper layer of the bridge portion 130, such that the air leak 146 is in fluidic communication with the upper channel of the bridge portion 130. Upon the application of suction to the conduit 108, gas (such, as air) can enter through the gas leak 146 and move from the proximal end of the bridge portion 130 to the distal end of the bridge portion along the upper channel of the bridge portion 130. The gas can then be suctioned into the lower channel of the bridge portion 130 by passing through the apertures through the distal ends of the upper, intermediate, and lower layers.

The air leak 146 can include a filter. Preferably, the air leak 146 is located at the proximal end of the bridge portion 130 so as to minimize the likelihood of wound exudate or other fluids coming into contact and possibly occluding or interfering with the air leak 146 or the filter. In some instances, the filter can be a microporous membrane capable of excluding microorganisms and bacteria, and which may be able to filter out particles larger than 45 μm. Preferably, the filter can exclude particles larger than 1.0 μm, and more preferably, particles larger than 0.2 μm. Advantageously, some implementations can provide for a filter that is at least partially chemically-resistant, for example to water, common household liquids such as shampoos, and other surfactants. In some cases, reapplication of vacuum to the suction adapter or wiping of the exposed outer portion of the filter may be sufficient to clear any foreign substance occluding the filter. The filter can be composed of a suitably-resistant polymer such as acrylic, polyethersulfone, or polytetrafluoroethylene, and can be oleophobic or hydrophobic. In some cases, the gas leak 146 can supply a relatively constant gas flow that does not appreciably increase as additional negative pressure is applied to the conduit 108. In instances of the negative pressure wound treatment system 100 where the gas flow through the gas leak 146 increases as additional negative pressure is applied, preferably this increased gas flow will be minimized and not increase in proportion to the negative pressure applied thereto. Further description of such bridges, conduits, air leaks, and other components, features, and details that can be used with any implementations of the negative pressure wound treatment systems disclosed herein are found in U.S. Pat. No. 8,801,685, which is incorporated by reference in its entirety as if fully set forth herein.

Any of the wound therapy devices (such as, the device 110 or 110′) disclosed herein can provide continuous or intermittent negative pressure therapy. Continuous therapy can be delivered at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. Intermittent therapy can be delivered between low and high negative pressure set points (sometimes referred to as setpoint). Low set point can be set at above 0 mmHg, −25 mmHg, −40 mmHg, −50 mmHg, −60 mmHg, −70 mmHg, −80 mmHg, −90 mmHg, −100 mmHg, −120 mmHg, −140 mmHg, −160 mmHg, −180 mmHg, or below −180 mmHg. High set point can be set at above −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. During intermittent therapy, negative pressure at low set point can be delivered for a first time duration, and upon expiration of the first time duration, negative pressure at high set point can be delivered for a second time duration. Upon expiration of the second time duration, negative pressure at low set point can be delivered. The first and second time durations can be same or different values.

In operation, the wound filler 102 can be inserted into the cavity of the wound 104, and wound cover 106 can be placed so as to seal the wound 104. The wound therapy device 110 can provide negative pressure to the wound cover 106, which can be transmitted to the wound 104 via the wound filler 102. Fluid (such as, wound exudate) can be drawn through the conduit 108 and stored in a canister. In some cases, fluid is absorbed by the wound filler 102 or one or more absorbent layers (not shown).

Wound dressings that can be utilized with the pump assembly and systems of the present application include Renasys-F, Renasys-G, Renasys AB, and Pico Dressings available from Smith & Nephew. Further description of such wound dressings and other components of a negative pressure wound therapy system that can be used with the pump assembly and systems of the present application are found in U.S. Patent Publication Nos. 2012/0116334, 2011/0213287, 2011/0282309, 2012/0136325 and U.S. Pat. No. 9,084,845, each of which is incorporated by reference in its entirety as if fully set forth herein. In some cases, other suitable wound dressings can be utilized.

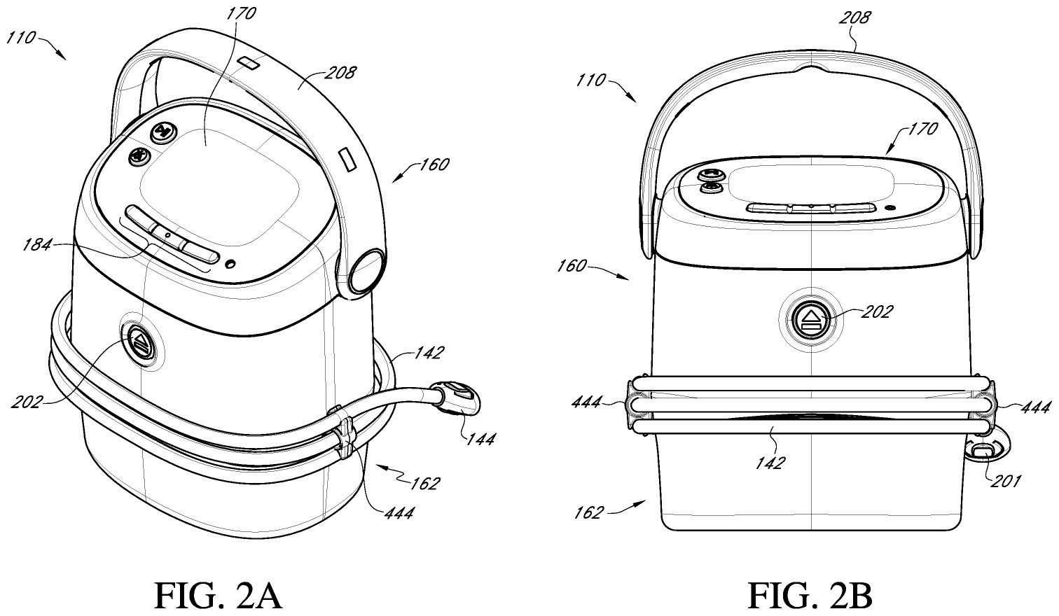

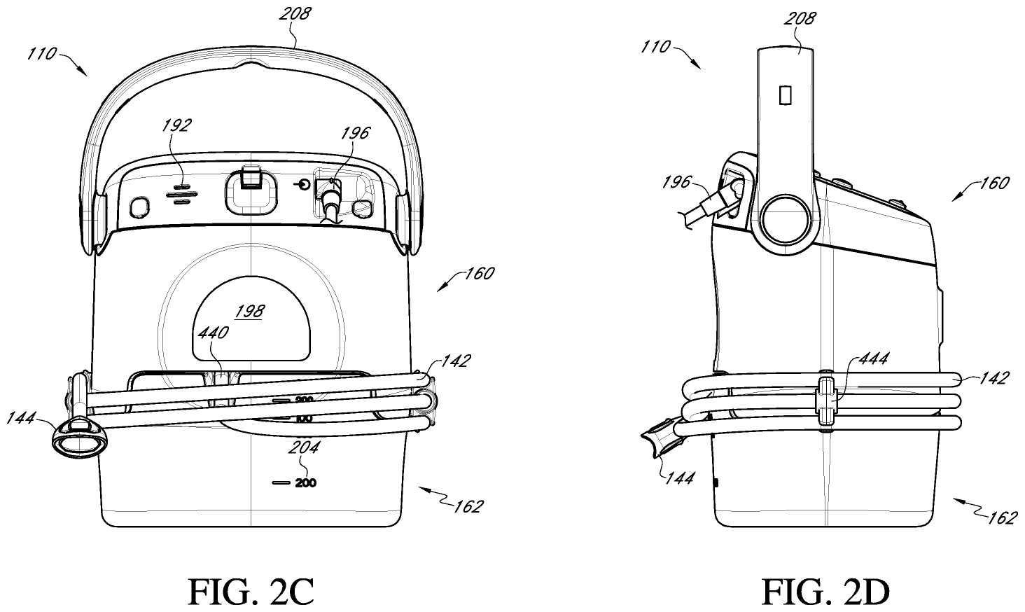

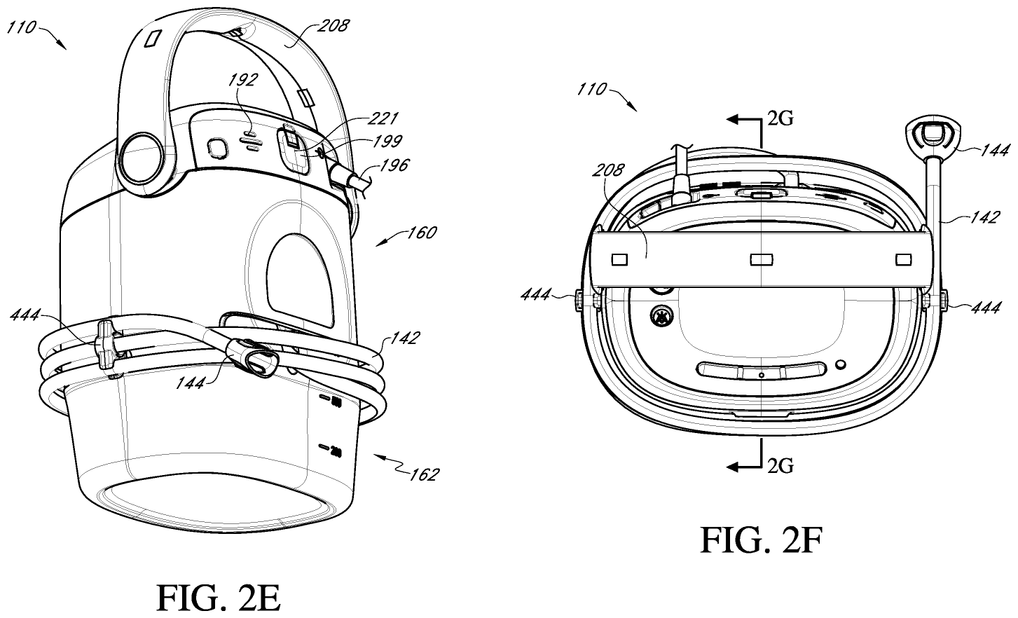

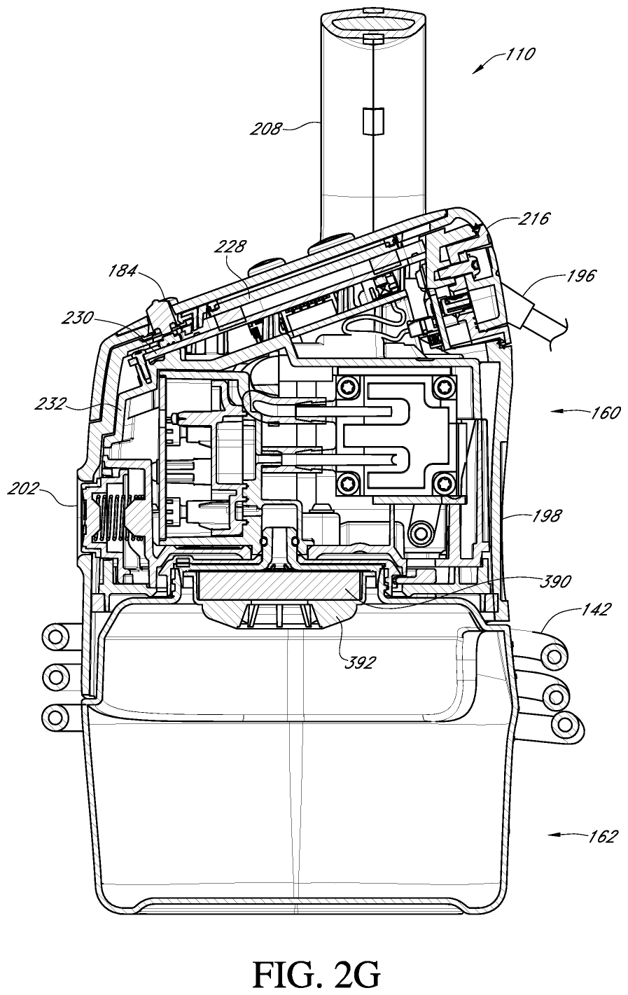

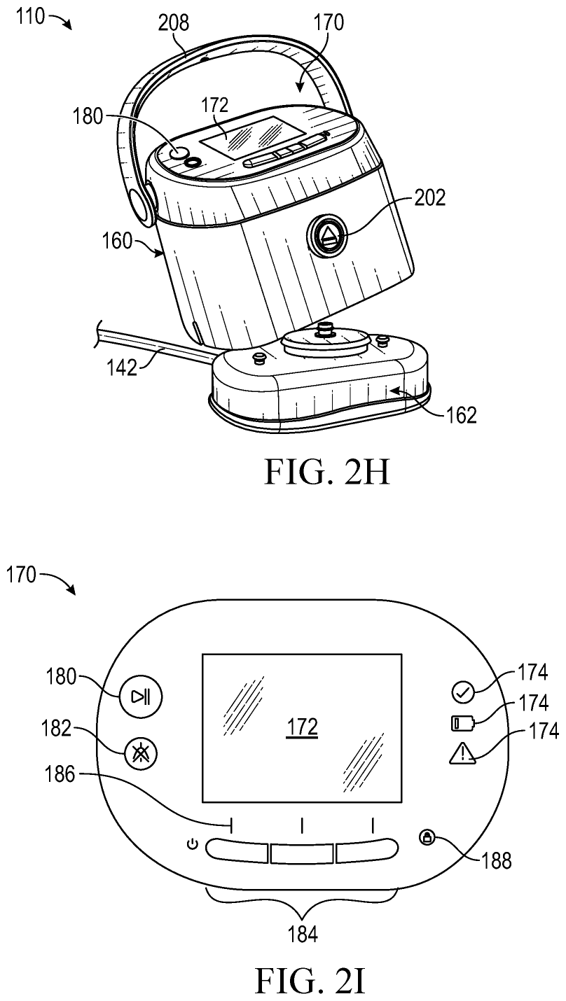

FIGS. 2A-2I show an arrangement of the negative pressure wound therapy device 110. In some arrangements, as will be described, the negative pressure wound therapy device 110 can have a pump assembly 160 that has a modular design, in that many of the subcomponents of the pump assembly 160 are grouped and designed to be in modules. This modular arrangement of the various components of the pump assembly 160 can make it easier and quicker to remove and replace any failed components of the pump assembly 160.

As illustrated, the pump assembly 160 and canister 162 can be connected, thereby forming the wound therapy device 110. The pump assembly 160 can include a user interface, communications interfaces, negative pressure generation and control, and alarms generation. Some arrangements of the pump assembly 160 can include a housing, the main function of which is to enclose or house the electronics and other components. The housing can provide patient safety and isolation from the device internals, can protect the pump device against impact damage, and can provide aesthetic appeal. The housing can also provide a clear window to allow the user to view the display. The main housing can be easily removed without disturbing the rest of the device and component connections (i.e., can be removed without requiring any electrical connectors to be disconnected), making for simpler repairs.

The pump assembly 160 can also include a core assembly (such as core assembly 212), which is also referred to as a core module. Some arrangements of the core assembly can include all or mostly all of the electrical and mechanical components and features required for the user interface, negative pressure control, and battery operation. In some arrangements, the core assembly can be the central sub-assembly of the pump assembly and can be configured to be an easily extractable part from the pump assembly to make service and repair easier and faster.

The core assembly can be separated out into separate components easily for ease of service, cleaning, and assembly. The pump assembly 160 can also include a rear trim panel, which can provide a USB interface, a charger port, and a speaker. The rear trim panel can be easily removed (e.g., by removing two screws and associated screw cover plates) to allow for quick repairs to the charger connector, which can be broken through device misuse. The pump assembly 160 can also include a handle or carrying strap, allowing for easy device portability.

With reference to FIG. 21, the pump assembly 160 can include an interface panel 170 having a display 172, one or more indicators 174, or one or more controls or buttons, including, for example and without limitation, a therapy start and pause button 180 or an alarm/alert mute button 182. Some arrangements of the lens of the display can be made from polycarbonate with a hard coat layer, or from poly methylmethacrylate. The interface panel 170 can have one or more input controls or buttons 184 (three being shown) that can be used to control any functions of the pump assembly 160 or the interface panel 170. For example and without limitation, one or more of the buttons 184 can be used to turn the pump assembly 160 on or off, to start or pause therapy, to operate and monitor the operation of the pump assembly 160, to scroll through menus displayed on the display 172, or to control or perform other functions. In some cases, the command buttons 184 can be programmable, and can be made from a tactile, soft rubber.

In some arrangements, the interface panel 170 can be generally planar and can be angled slightly toward a front surface of the pump assembly 160 relative to a horizontal plane. For example and without limitation, some arrangements of the interface panel 170 can be angled forward by 20° (or approximately 20°), or from 15° (or approximately 10°, or less than 10°) to 30° (or approximately 30°, or more than 30°), or by any angle within the foregoing range, relative to a horizontal plane.

The interface panel 170 can have visual indicators 186 that can indicate which of the one or more buttons 184 is active. The interface panel 170 can also have a lock/unlock control or button 188 that can be configured to selectively lock or unlock the functionality of the various buttons (e.g., buttons 184) or the display 172. In some arrangements, when the lock/unlock button 188 is in the locked state, depressing one or more of the various other buttons or the display will not cause the pump assembly 160 to change any display functions or performance functions of the device. This way, the interface panel 170 will be protected from inadvertent bumping or touching of the various buttons or display. In some arrangements, the interface panel 170 can be configured such that, while the device and/or the interface panel 170 is in a locked state, one or more of the buttons on the interface panel 170 can be used, but adjustments to the therapy settings of the device will be blocked or prevented in the locked state. The interface panel 170 can be located on an upper portion of the pump assembly 160, for example and without limitation on an upward facing surface of the pump assembly 160.

The display 172, which can be a screen such as an LCD screen, can be mounted in a middle portion of the interface panel 170. The display 172 can be a touch screen display. The display 172 can support playback of audiovisual (AV) content, such as instructional videos, and render a number of screens or graphical user interfaces (GUIs) for configuring, controlling, and monitoring the operation of the pump assembly 160.

The one or more indicators 174 can be lights (such as, LEDs) and can be configured to provide a visual indication of alarm conditions and or a status of the pump. For example and without limitation, the one or more indicators 174 can be configured to provide a visual indication of a status of the pump assembly 160 or other components of the negative pressure wound treatment system 100, including without limitation the conduit 108 or the wound cover 106 (such as, to provide an indication of normal operation, low battery, a leak, canister full, blockage, overpressure, or the like). For example and without limitation, the one or more indicators 174 can indicate to a user (for example, patient, health care provider, or the like) a variety of operating or failure conditions of the pump assembly 160, including alerting the user to normal or proper operating conditions, pump failure, power supplied to the pump or power failure, detection of a leak within the wound cover or flow pathway (sometimes referred to as fluid flow path), suction blockage in the flow pathway, canister full, overpressure, and/or any other similar or suitable conditions or combinations thereof. For example and without limitation, the one or more indicators 174 can indicate to a user (for example, patient, health care provider, or the like) a system okay status, a status of the level charge of a battery or that the batteries are actively being charged, and/or an alarm or alert status. Any one or more suitable indicators can be additionally or alternatively used, such as visual, audio, tactile indicator, and so on.

FIG. 2C shows a back or rear view of the wound therapy device 110 shown in the FIG. 2A. As shown, the pump assembly 160 can include a speaker 192 for producing sound. For example and without limitation, the speaker 192 can generate an acoustic alarm in response to deviations in therapy delivery, non-compliance with therapy delivery, or any other similar or suitable conditions or combinations thereof. The speaker 192 can provide audio to accompany one or more instructional videos that can be displayed on the display 172.

The pump assembly 160 can be configured to provide easy access (such as, an access door on the casing of the pump assembly) to one or more filters of the pump assembly 160, such as antibacterial filters. This can enable a user (such as, a healthcare provider or patient) to more easily access, inspect or replace such filters. The pump assembly 160 can also include a power jack assembly 196 (that can include the power cord) for providing power to the pump assembly 160 or for charging and recharging an internal power source (such as, a battery). Some implementations of the pump assembly 160 can include a disposable or renewable power source, such as one or more batteries, so that no power jack is needed. The pump assembly 160 can have a recess 198 formed therein to facilitate gripping of the pump assembly 160.

The canister 162 can hold fluid aspirated from the wound 104. For example, the canister 162 can have a canister body 346 having an 800 mL (or approximately 800 mL) capacity, or from a 300 mL or less capacity to a 1000 mL or more capacity, or any capacity level in this range. The canister 162 can include a tubing for connecting the canister body 346 to the conduit 108 in order to form a fluid flow path. The canister 162 can be replaced with another canister, such as when the canister 162 has been filled with fluid or when the user has finished her or his treatment.

In some arrangements, the canister 162 can include a low-cost disposable assembly that stores exudate that has been extracted from the wound. The canister 162 can be non-sterile and can be designed for a single-use that can be disposed of after collection of exudate from a single user. The canister 162 will collect wound exudate and can be available with and without solidifier. The solidifier can solidify the exudate collected in the canister. The canister 162 can be secured under the pump assembly 160 using a locking mechanism that will be described.

With reference to FIG. 2F, the wound therapy device 110 can include a canister inlet tube 142 in fluid communication with the canister body 346 of the canister 162. The canister inlet tube 142 can be coupled with a dressing port connector 144 that can be used to connect with the conduit 108.

The canister 162 can be selectively coupleable and removable from the pump assembly 160. With reference to FIG. 2H, in some cases, a canister release button 202 can be pushed to selectively release the canister 162 from the pump assembly 160. With reference to FIG. 2C, the canister 162 can have one or more fill lines or graduations 204 to indicate to the user and amount of fluid or exudate stored within the canister 162.

The wound therapy device 110 can have a handle 208 that can be used to lift or carry the wound therapy device 110. The handle 208 can be coupled with the pump assembly 160 and can be rotatable relative to the wound therapy device 110 so that the handle can be rotated upward for lifting or carrying the wound therapy device 110 or the pump assembly 160, or rotated into a lower profile in a more compact position when the handle is not being used. In some cases, the handle 208 can be coupled with the pump assembly 160 in a fixed position. The handle 208 can be coupled with an upper portion of the pump assembly 160 or can be removable from the wound therapy device 110.

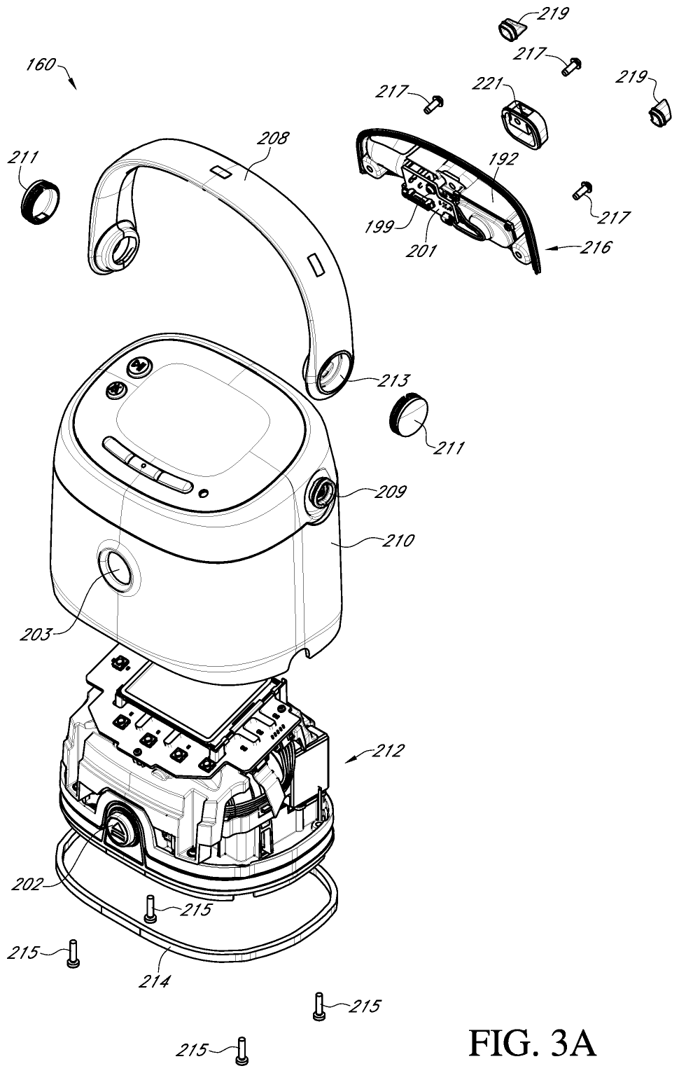

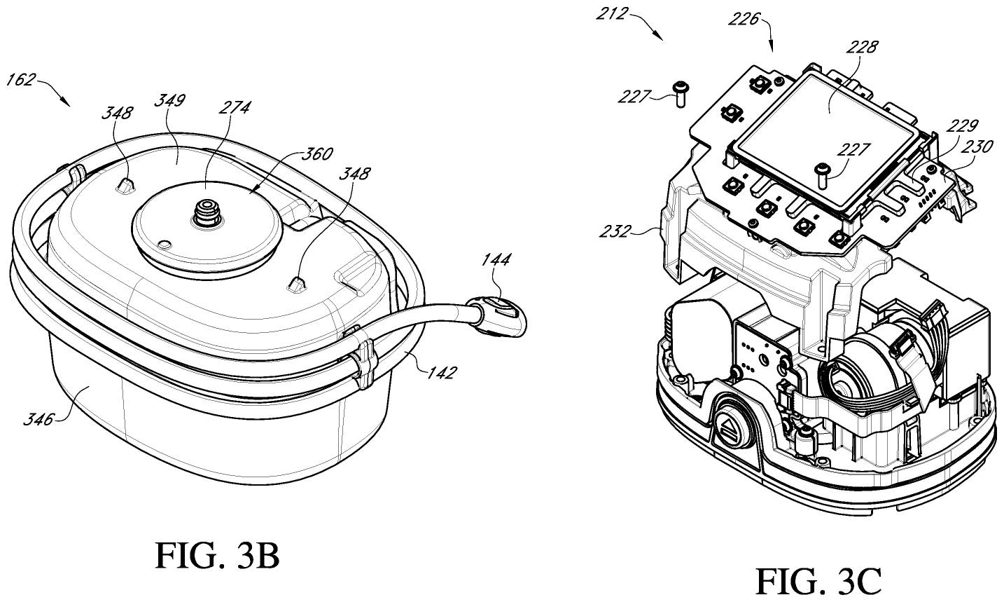

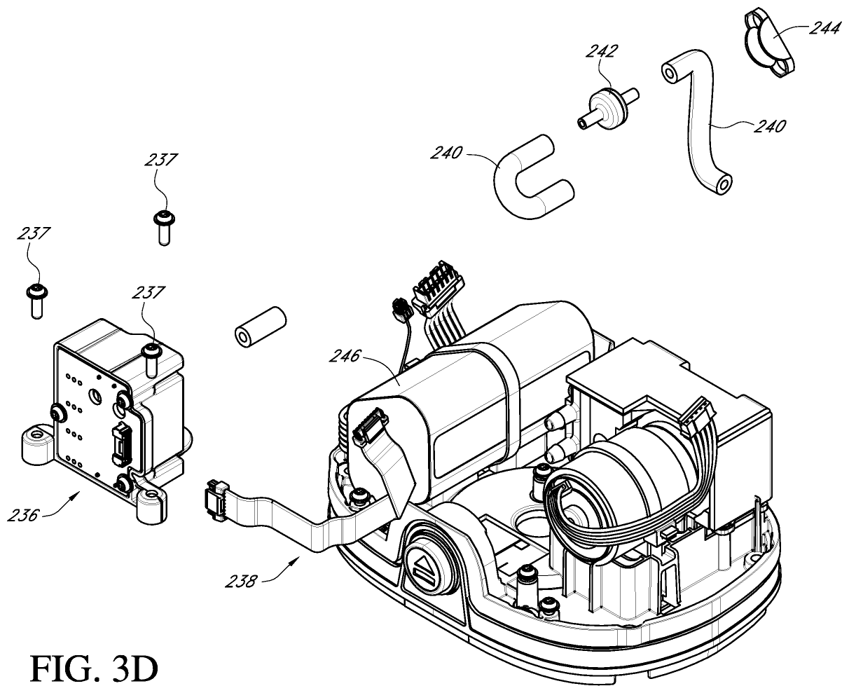

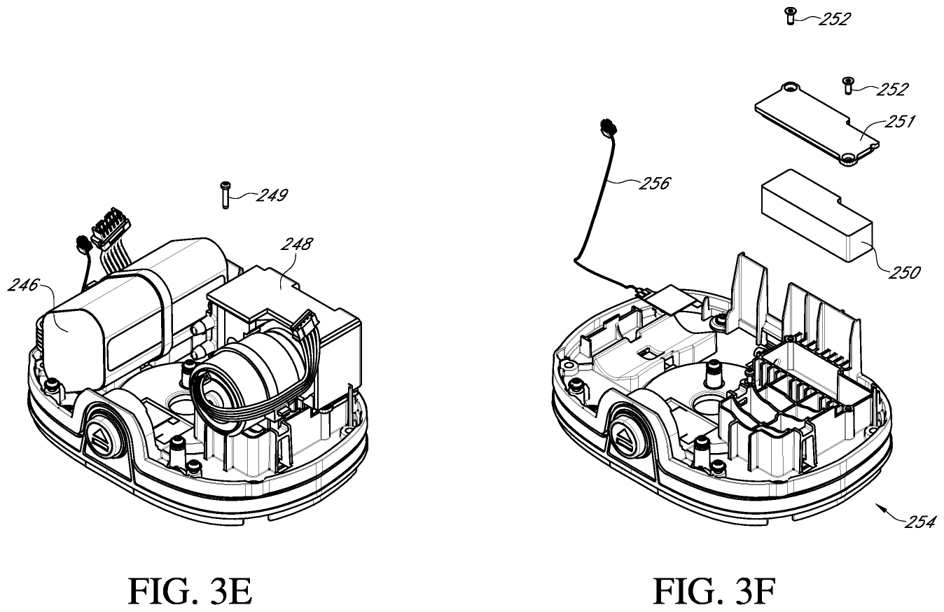

FIG. 3A is an exploded view of the pump assembly 160. Some arrangements of the pump assembly 160 have been designed to facilitate the ease with which components and sub-components of the pump assembly 160 can be removed from the pump assembly 160 for cleaning, servicing, replacing, or otherwise. As will be described, some arrangements of the pump assembly 160 are designed such that the various components and/or sub-assemblies are arranged and assembled in the pump assembly 160 in a modular fashion to make the various components and sub-assemblies easier to remove, clean, and/or service. In some arrangements, the pump assembly 160 can have a housing 210 which is sized and configured to enclose at least a core assembly 212. The housing 210 can be made from acrylonitrile butadiene styrene, or any other desirable or suitable materials.

A core base seal 214 can be used to seal the collection canister 162 to the bottom of the pump assembly 160. A plurality of screws or other fasteners 215 can be used to couple the core assembly 212 with the housing 210. A rear trim assembly 216 can be coupled with the housing 210 using one or more fasteners 217 that can have one or more screw or fastener covers 219. Some arrangements of the rear trim assembly 216 can have a USB port 199 or other wired connection ports coupled with a printed circuit board 201 that can be included in the rear trim assembly 216. The rear trim assembly 216 can include the speaker 192 and can have other components or connectors, buttons, switches, or inputs. A USB cover 221 can be removably coupled with the USB port.

In some arrangements, the power cord 196 can be part of a power jack assembly, and/or can be directly connected to the rear trim assembly 216. The power cord 196 or other components of the power jack assembly or other components that are in electrical communication with the power cord 196 can extend through the rear trim assembly 216 and be electrically coupled with electrical components of the rear trim assembly 216 and/or other components of the pump assembly 160. In some arrangements, the power jack assembly, which can include the power cord 196, can be connected to the rear trim assembly 216. The power jack assembly and the pump device 160 can be configured, in some arrangements, such that the power cord 196 can be easier to remove and replace as compared to models in which the power cord is connected directly to the internal electronic components within the pump, which can require that the service technician disassemble the pump to replace the power cord, possibly remove and make new soldered connections, etc. In some arrangements of the pump assembly 160, the service technician need only remove the power jack assembly from the rear trim assembly 216 and install a new power jack assembly to replace the power cord 196, or, in some arrangements, remove and replace the rear trim assembly 216 that the power jack assembly and/or the power cord 196 is attached to replace the power cord 196.

The handle 208 can be coupled with posts 209 of the housing 210 configured to pivot relative to the housing 210. In some arrangements, the handle can be made from a thermoplastic elastomer or any other suitable or desired material. In some arrangements, the handle can be configured to simply clip or snap onto the housing 210. In some arrangements, the handle 208 can be configured to rotate relative to the housing 210, or can be rigidly (nonrotatably) attached to the housing 210. In some arrangements, two or more handle caps 211 can be used to couple the handle 208 with the posts 209 of the housing 210 or can be used to cover the depressions or recesses 213 in the handle 208. The canister release button 202 can extend through an opening 203 in the housing 210.

With reference to FIG. 3C, core assembly 212 can include an upper core assembly 226 that can be coupled with the core assembly 212 using one or more, or two or more fasteners 227, which can be screws. The upper core assembly 226 can include a display assembly 228 that can have a 2.8 inch TFT display, or a 3 inch display, or any other suitable display. The upper core assembly 226 can include a foam spacer 229 between the display assembly 228 and a printed circuit board 230 that can include a processor, a memory device, and other electronic components for operating the display assembly 228 and the pump assembly 160. One or more cable connectors can be used to electronically couple the display assembly 228, the printed circuit board 230, and the other components of the core assembly 212. Additionally, the one or more buttons and/or indicators 174, 180, 182, 184, 194 and/or other buttons or indicators can be electronically and/or physically coupled with the printed circuit board 230. The display assembly 228, the foam spacer 229, and the printed circuit board 230 can be supported by a support base 232 (also referred to herein as an upper support) that can be coupled with the other components of the core assembly 212 using the fasteners 227 such that the components of the upper core assembly 226 and that are related to the display of the pump assembly 160 can be removed from the pump assembly 160 by removing the support base 232 and, in some arrangements, the cable connectors associated with the components of the upper core assembly 226 from the other components for the core assembly 212.

With reference to FIG. 3D, the core assembly 212 can include a flow module 236 (or a flow manifold) that can be configured to receive the air/fluid that is drawn through the collection canister 162. For example and without limitation, the flow module 236 can have an opening 233 or port that can be aligned with an opening 235 in the lower support 264 of the lower core assembly 254 and/or a connector interface 372 of the collection canister 162 so that air/fluid drawn through the opening 373 extending through the connector interface 372 can be drawn through the opening 233 in the flow module 236 by the pump module 248. In this arrangement, the pump module 248 can draw air/fluid through the flow module 236 (positioned upstream of the pump module 248), through a tube or conduit 241 in communication with an exit opening or port of the flow module 236 and in communication with a tubing connector 247 (e.g., a first tubing connector 247a) of the pump module 248. In any arrangements disclosed herein, the tubing can have a ⅛ inch inner diameter.

In some arrangements, the flow module 236 can be coupled with the other components of the core assembly 212 using one or more fasteners 237 (three being shown). The flow module 236 can be electronically coupled with the other components of the core assembly 212 using a flow module wire strip 238. Two or more flow tubes, or three or more flow tubes can be used to provide a fluid flow path from the pump module 248 to the canister or other components, as described above and below. Some arrangements of the flow module 236 can include a pressure sensor, or multiple pressure sensors. In some arrangements, a flow check valve 242 can be used to prevent a flow of air into the canister from the pump. In some arrangements, the check valve 242 can have barbed connectors for connecting with tubing and can be made from any suitable material or materials, including, without limitation, acrylic and silicone.

The check valve 242 can be coupled with the tubing or conduit 240 (e.g., a first conduit 240a) that is coupled with a tubing connector 247 (e.g., a second tubing connector 247b) of the pump module 248. A second conduit 240b can be coupled with a downstream connector or side of the check valve 242 to communicate the air/fluid passing through the check valve 242 to an exhaust chamber 250 (also referred to herein as a noise reduction chamber or a first noise reduction chamber), as described in greater detail below. For example and without limitation, the second conduit 240b can be coupled with an inlet or opening 255 of the exhaust chamber 250 to communicate the air/fluid advanced through the check valve 242 to an exhaust chamber 250, which will be described in greater detail below. A tubing clip 244 can be used to retain the one or more pieces of tubing 240 to the core assembly 212.

With reference to FIG. 3E, the core assembly 212 can include a battery module 246 and a pump module 248. A cover 249 can cover one or more sides of the pump module 248. For example and without limitation, the cover 249 can cover the top, back, and sides of the pump module 248. The pump module 248 includes a pump motor. The pump module 248 can include tubing connectors 247 (that can include an inlet tubing connector and an outlet tubing connector) to which tubing can be coupled.

The pump can be a diaphragm pump or any other desired or suitable type of pump, including a rotary pump, a peristaltic pump, a piezoelectric pump, or otherwise. Some arrangements of diaphragm pumps are well suited to the flow rates and pressures required, have a long maintenance-free service life, and are relatively efficient and quiet in operation.

In any arrangements disclosed herein, the pump module 248 can be oil-less, can be characterized by low power consumption and low sound level, and can have a compact and lightweight design. The pump module 248 can have a 12V or 24V motor, can have a maximum flow rating of 4 liters/min, approximately 4 liters/min, 3 liters/min, or approximately 3 liters/min, and can have a max intermittent pressure of 1.9 bars. Some arrangements of the pump module 248 can include a pump controller. In some arrangements, the pump module 248 can be a diaphragm pump and compressor type pump.

The battery module 246 can be any suitable battery pack and can include single use and rechargeable type batteries such as lithium ion batteries. Some arrangements of the battery can include lithium ion 18650 cells, or any other type of battery that has sufficient or plentiful power supply, has good power density for size, and/or is lightweight, though none of these features or characteristics is required. The battery module can include a carefully designed charging circuitry with full redundancy due to the inherent risks of lithium ion battery technology, and can be configured to operate across a limited temperature range. The battery module 246 and/or the pump module 248 can be coupled with or supported by a lower core assembly 254. One or more fasteners 249 and/or cable ties can be used to couple the pump module 248 with the other components of the core assembly 212.

With reference to FIG. 3F, the core assembly 212 can include an exhaust chamber 250 configured to reduce the sound level or noise produced by the pump module 248. In some arrangements, a cover plate 251 can be secured to the exhaust chamber 250 using one or more fasteners 252 (two being shown). A foam element 253 can be positioned within the exhaust chamber 250. The foam element 253 can be sized to fit snuggly or tightly within the exhaust chamber 250 and can be configured to attenuate a noise level of the pump module 248 and/or the exhaust air from the pump module 248. For example and without limitation, in some arrangements, the foam element 253 can have a size and a shape that is similar to or the same as a size and a shape of the space within the exhaust chamber 250 or, in some arrangements, the foam element 253 can have a shape that is similar to or the same as the shape of the space within the exhaust chamber 250 and a size that is oversized as compared to the exhaust chamber 250 (e.g., 10% bigger by volume or dimensionally, or from 5% bigger to 20% bigger by volume or dimensionally). With reference to FIGS. 3F and 3J, the exhaust chamber 250 can have an inlet 255 and an outlet 257. The inlet can include a nipple or port for receiving a tubing or conduit component. The outlet 257 can essentially allow the exhaust gas from the pump module 248 to exit the pump assembly 110.

Some arrangements of the pump assembly can have a noise reduction element in the exhaust flow, in addition to the exhaust chamber 250 or without the exhaust chamber 250. In some arrangements, the noise reduction element can be or can include an air pulse reduction chamber 460 (also referred to herein as a noise reduction chamber) downstream of the pump module 248, as shown in FIGS. 3G-3K. With reference to FIGS. 3G-3K, the noise reduction chamber 460 can be coupled with a tubing or conduit 462 to a tubing connector 247 (e.g., the second tubing connector 247b) of the pump module 248 so that the air/fluid exiting the pump module 248 then passes through the noise reduction chamber 460 before exiting the pump assembly 160. The conduit 462 can be coupled with an intake opening or port 464 of the air pulse reduction chamber 460. A second tubing or conduit 466 can be coupled with an outtake opening or port 468 of the air pulse reduction chamber 460 such that air/fluid enters the intake opening 464 of the air pulse reduction chamber 460 and exits the air pulse reduction chamber 460 through the outtake opening 468 of the air pulse reduction chamber 460. In some arrangements, the air/fluid exiting the air pulse reduction chamber 460 can then be communicated through the tubing 466 through a check valve 470. In some arrangements, the check valve 470 can be the same as or similar to the check valve 242, and vice-versa. A tubing or conduit 472 can be coupled with a tubing or conduit 476 using a connector 474 (which can be barbed) to communicate the air/fluid from the check valve 470 to the exhaust chamber 250.

With reference to FIGS. 3H and 3K, the air pulse reduction chamber 460 can have a main body 480 having the ports 464, 468, a cover or plate 482 coupled with an open side of the body 480, and a foam layer 484. The foam layer 484 is optional such that some arrangements do not have the foam layer 484. The cover 482 can be sealingly coupled with the body 480 of the air pulse reduction chamber 460. In some arrangements, the main body 480 can be formed so that a separate cover 482 is not required (e.g., wherein the body 480 and the cover 482 are integrally formed, such as by blow molding, 3D printing, or other suitable manufacturing methods). The foam layer 484 can be configured to dampen sound pulses from the air (or, more generally, the gas) traveling through the air pulse reduction chamber 460 and/or vibrations in the air pulse reduction chamber 460 to reduce noise and/or stresses on the air pulse reduction chamber 460. The body 480 can have a space 481 within the body through which the air/fluid entering the inlet opening 464 must pass before exiting the outlet opening 468.

With reference to FIG. 3K, some arrangements of the main body 480 of the air pulse reduction chamber 460 can have a first inner wall segment 490 that can be positioned adjacent to the inlet opening 464. The first inner wall segment 490 can have a height that is equal to or similar to a height of the perimeter walls 483 of the body 480. The first inner wall segment 490 can extend across a portion (e.g., a majority) of a distance across the space 481 so the air/fluid entering the inlet opening 464 must pass through an opening/passageway 492 between an end of the first inner wall segment 490 and the outer perimeter wall 483 before passing though the outlet opening 468. Therefore, the first inner wall segment 490 acts like a flow deflector or diverter or a fin of a baffle to attenuate a magnitude of pulses of air from the pump module 248 (which can have a diaphragm pump) flowing through the air pulse reduction chamber 460. Some arrangements of the main body 480 of the air pulse reduction chamber 460 can have a recess 494 that can add additional volume to the main body 480 of the air pulse reduction chamber 460 to further attenuate a magnitude of pulses of air flowing through the air pulse reduction chamber 460. Attenuating the magnitude of pulses of air flowing through the air pulse reduction chamber 460 can reduce the magnitude of air pulses imparted on the check valve 470 downstream of the air pulse reduction chamber 460 to quiet the check valve 470 during operation of the pump assembly 160. Some arrangements of the main body 480 can have a second inner wall segment (not shown) or three or more inner wall segments sized and positioned to block or deflect a passage of air through the air pulse reduction chamber 460.

In some arrangements, the pump module 248 can be configured to exhaust the air that is drawn through the collection canister 162 through an exhaust port (such as through exhaust outlet or port 257), or one or more exhaust ports. In some arrangements, such as in the illustrated arrangement, the pump module 248 can be configured to exhaust the air that is drawn through the collection canister 162 through one or more spaces, gaps, cracks, or other openings formed in the housing 210 such that the pump assembly 160 does not have a discrete exhaust port on the exterior of the housing 210. This can be achieved in some arrangements because the canister 162 can have an odor filter integrated therein so that any substances or vapors that may be in the exhaust air are filtered and removed, or substantially removed, by an odor filter in the canister before the exhaust air reaches the intake in the pump assembly 160.

Other details related to the exhaust vent or exhaust of the pump assembly which can be used with any arrangements of the negative pressure wound treatment system 100 disclosed herein are set forth in international application WO 2019/211732, published on Nov. 7, 2019, which is hereby incorporated by reference as if fully set forth herein.

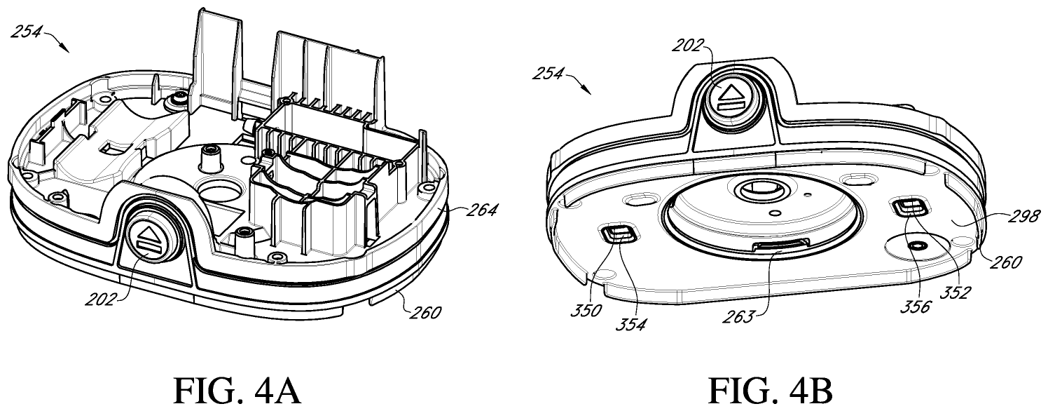

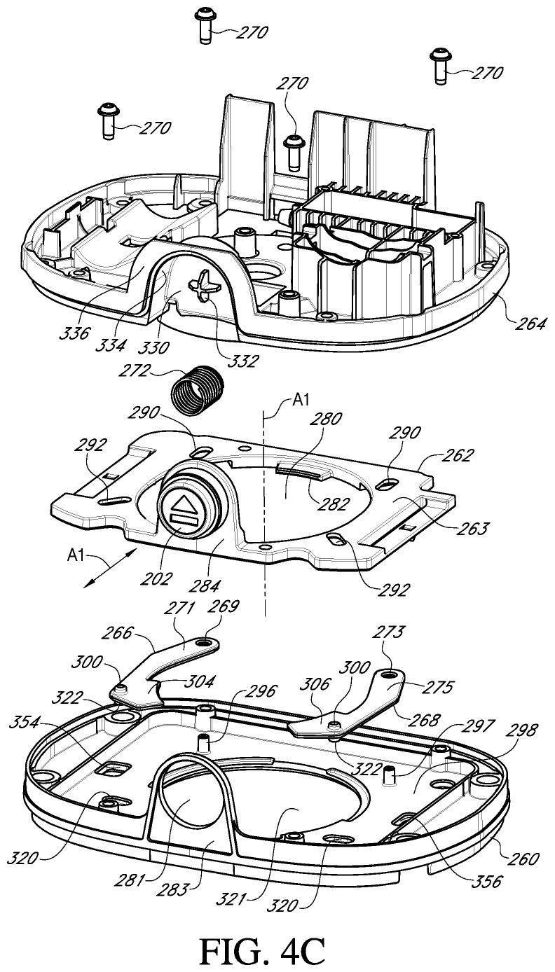

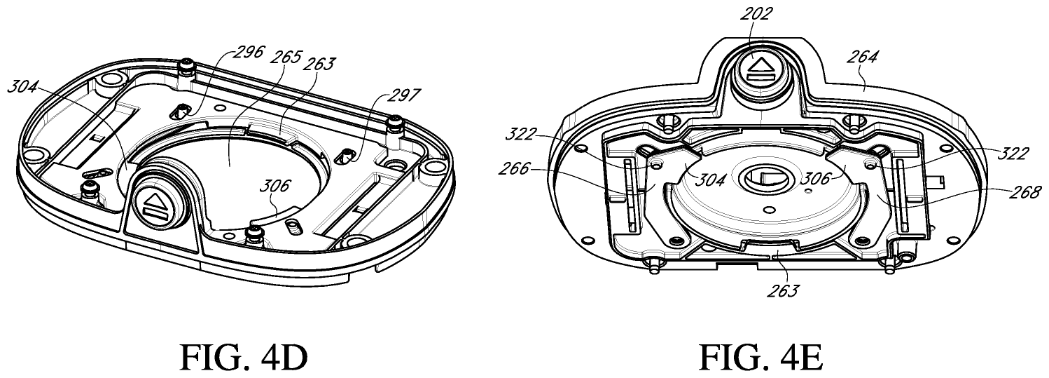

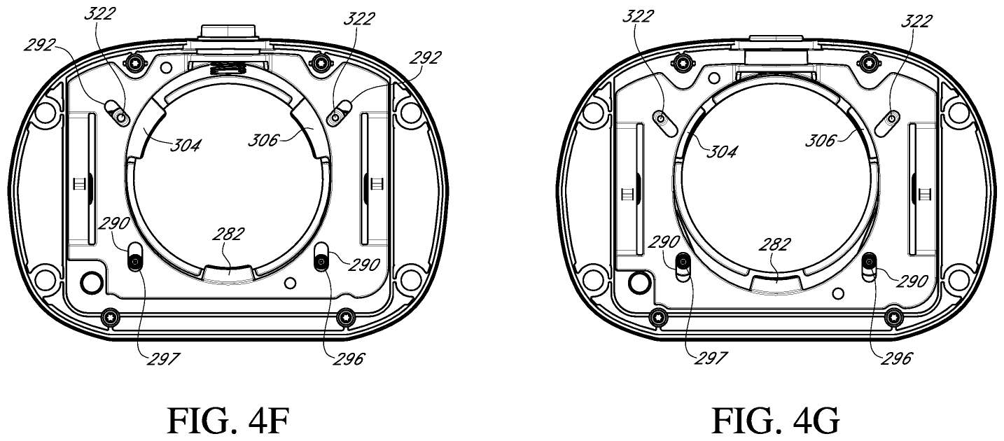

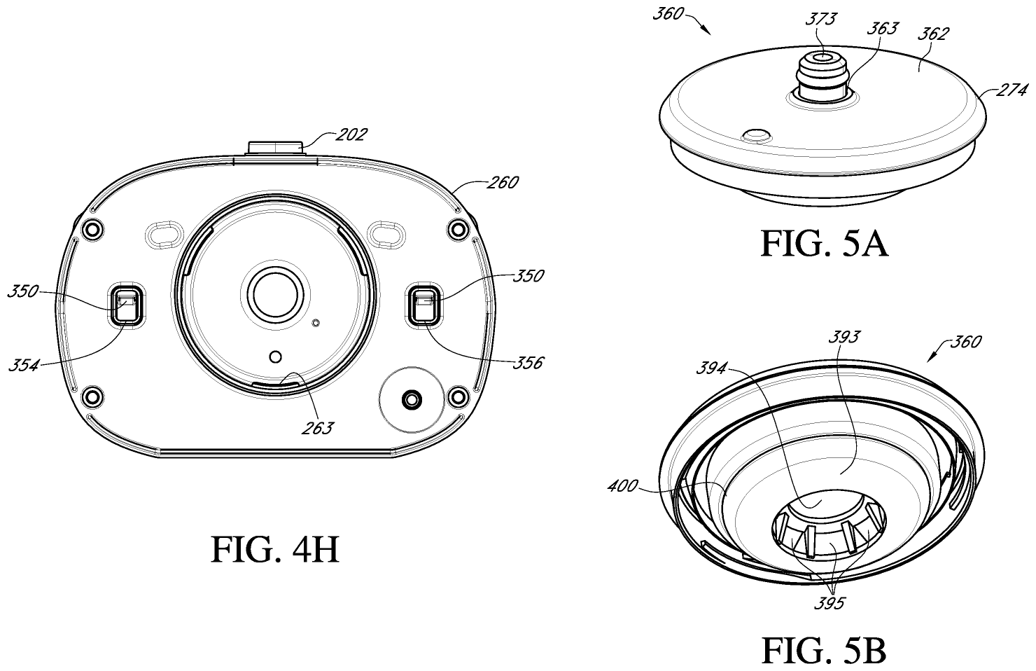

FIGS. 4A-4H show an arrangement of the lower core assembly 254. The lower core assembly 254 can have a base support 260, a second support 264 (also referred to herein as a lower support), and a canister release mechanism. In some arrangements, the canister release mechanism can include an actuator 262, a first locking arm 266, and a second locking arm 268. In some arrangements, the button 202 can be coupled with or integrally formed with the actuator 262. In addition to supporting the pump module and battery, the lower core assembly 254 can be configured to support a selectively movable locking mechanism that can selectively couple the canister 162 with the pump assembly 160.

One or more fasteners 270 (four being shown) can be used to couple the second support 264 with the base support 216. The actuator 262 can be positioned between the base support 260 and a second support 264 can be slidable relative to the base support 260 and the second support 264 between a first position and a second position. In some arrangements, the actuator 262 can move along a first axis A1 between the first and second positions. The first axis A1 can be generally parallel with an axial centerline of the button 202 and/or of the spring 272. In the first position, actuator 262 can be in a locking or engaged position with respect to an upper portion of the canister 162, such as a neck flange or neck portion 274 of the canister 162 (as shown in FIG. 3B) such that the canister 162 will be securely engaged with the lower core assembly 254 of the pump assembly 160 when the actuator 262 is in the first position. The first position of the actuator 262 is shown in FIGS. 4A, 4D, 4E, and 4F, for example and without limitation. In the second position, actuator 262 can be retracted or disengaged from the neck flange or neck portion 274 of the canister 162 such that the canister 162 can be freely removed from the lower core assembly 254 and, hence, the pump assembly 160. The second position of the actuator 262 is shown in FIGS. 4G, for example and without limitation.

The actuator 262 can be moved from the first position to the second position pushing the canister release button 202 or moving the canister release button 202 toward the second support 264. A spring or other resilient member 272 can be positioned between a portion of the actuator 262 (for example and without limitation, adjacent to the canister release button 202) and the second support 264 to bias the actuator 262 toward the first position. In this configuration, releasing the canister release button 202 will cause the spring 272 to automatically move the actuator 262 from the second position to the first position if the actuator 262 is in the second position.

Some arrangements of the actuator 262 can have a base portion 263 and an opening 280 large enough to receive the neck portion 274 of the canister 162 (such as, for example and without limitation, when the actuator 262 is in the second position) extending through and generally perpendicular to the base portion 263. The actuator 262 can also have a projection or latch 282 that can extend into the opening 280 such that the latch 282 engages with the neck portion 274 when the actuator 262 is in the first position. The actuator 262 can be arranged such that the latch 282 moves between the first position and the second position when the actuator moves between the first position and a second position. The button 202 can extend away from a tab portion or flange 284 of the actuator 262 in a direction that is parallel with the first axis A1. The button 202 can extend or project in a direction that is generally perpendicular to an axial centerline A2 of the opening 280 in the actuator 262. With reference to FIG. 4C, the canister release button 202 can extend away from the opening 280 through an opening 281 formed in a tab portion of flange 283 projecting away from a base portion 298 of the base support 260. Additionally, the actuator 262 can be configured such that the actuator 262 moves between the first position on the second position in a direction that is generally perpendicular to the axial centerline A2 of the opening 280.

The actuator 262 can have a first set of slots 290 arranged generally in a direction that is generally parallel with the first axis A1. The actuator 262 can have a second set of slots 292 arranged at an angle relative to the direction of the first axis A1—for example, at an angle that is approximately 45° relative to the first axis A1, or from 40° to 50°, or from 30° to 60° relative to the first axis A1. The slots 290 can be configured to receive first and second projections 296, 297 extending from a base portion 298 of the base support 260. The length of the slots 290 can be long enough to permit the movement of the actuator 262 between the first and second positions before the projections 296, 297 inhibit the movement of the actuator 262.

The first locking arm 266 can have an opening 269 extending through a main body portion 271 of the first locking arm 266 that can be configured to receive the first projection 296 extending from the base support 260. The opening 269 can extend through the first locking arm 266 in a direction that is generally parallel with the axial centerline A2 of the opening 280 extending through the actuator 262. The first locking arm 266 can be configured to rotate about the first projection 296. For example, with the first projection 296 extending through the opening 269 in the first locking arm 266, the opening 269 can rotate about the first projection 296 as the first locking arm 266 rotates about the first projection 296.

Similarly, the second locking arm 268 can have an opening 273 extending through a main body portion 275 of the second locking arm 268 that can be configured to receive a second one of the projections 296 extending from the base support 260. The opening 273 can extend through the second locking arm 268 in a direction that is generally parallel with the axial centerline A2 of the opening 280 extending through the actuator 262. The second locking arm 268 can be configured to rotate about the second projection 297. For example, with the second projection 297 extending through the opening 273 in the second locking arm 268, the opening 273 can rotate about the second projection 297 as the second locking arm 268 rotates about the second projection 297.

The second set of slots 292 can be configured to receive projections 300, 302 extending from the main body portions 271, 275 of the first and second locking arms 266, 268, respectively. A length of the slots 292 can be sufficient to permit the movement of the projections 300, 302 of the first and second locking arms 266, 268 as the first and second locking arms 266, 268 rotate about the first and second projections 296, 297. The slots 292 can also limit a range of the movement of the first and second locking arms 266, 268 as the first and second locking arms 266, 268 rotate about the first and second projections 296, 297. The second set of slots 292 can be angled and configured to cause the first and second locking arms 266, 268 to rotate radially outwardly from the first position to the second position of the first and second locking arms 266, 268 by exerting a force on the projections 300 of the first and second locking arms 266, 268 as the actuator 262 is moved from the first position to the second position of the actuator 262.

The first and second locking arms 266, 268 can be configured to move or rotate between a first position and a second position. In the first position, a projection or latch 304 of the first locking arm 266 and a projection or latch 306 of the second locking arm 268 can overlap with and engage with the flange or neck portion 274 of the canister 162 or the cap assembly 360 (such as is shown in FIGS. 4D, 4E)—i.e., can overlap a radially extending surface 375 of the flange or neck portion 274 of the canister of the cap assembly 360. In the second position, the projection or latch 304 of the first locking arm 266 and the projection or latch 306 of the second locking arm 268 are disengaged from the flanged or neck portion 274 of the canister 162, as shown in FIG. 4D, 4G.

Some arrangements of the base support 260 can have slots 320 formed in the base portion 298 of the base support 260. The slots 320 can be configured to receive second projections 322 extending away from a second surface of the first and second locking arms 266, 268. The projections 322 extending away from the second surface of the first and second locking arms 266, 268 can extend in a direction that is opposite the direction that the projections 300 extend from the first and second locking arms 266, 268. The slots 320 can be configured to permit the projections 322 of the first and second locking arms 266, 268 to translate along the slots 320 as the first and second locking arms 266, 268 move between the first and second positions. Additionally, the base support 260 can have an opening 321 formed through the base portion 298 of the base support 260, the opening 321 extending generally in the direction that is parallel to the axial centerline A2 of the opening 280 of the actuator 262. The opening 321 can be large enough to receive the flanged or neck portion 274 of the canister 162 therein.

In some cases, the spring 272 can be positioned axially against a support surface 330 formed on a portion of the second support 264. A center projection 332 can also extend away from the support surface 330 to limit a movement of an end portion of the spring member 272 relative to the surface of the support surface 330. A recess 334 can be formed in the flanged portion 336 that is formed around the support surface 330. The recess 334 can be configured to receive the tab portion or flange 284 of the actuator 262 as the actuator 262 is moved from the first position to the second position.

Some arrangements of the canister 162 can have a canister body 346 having one or more angled projections 348 formed on an upper surface 349 of the canister body 346. The actuator 262 can have a first projection or tab 350 and a second projection or tab 352 that can extend through openings 354, 356, respectively, formed through the base portion 298 of the base support 260. The first and second rejections 350, 352 can extend in a direction that is generally parallel with the axial centerline A2 that extends through the opening 280 formed in the actuator 262. Projections can move from a first position to a second position as the actuator 262 first position to the second position of the actuator 262. The first and second projections 350, 352 can interact with a sloped or angled surface of the one or more angled projections 348 formed on the upper surface 349 of the canister 162. In some arrangements, as the first and second projections 350, 352 move along the angled surface of the angled projections 348, the first and second projections 350, 352 can exert a force on the angled projections 348 to cause the canister 162 to move away from the base support 260 of the lower core assembly 254 as the actuator 262 is moved from the first position to the second position. This can facilitate the removal of the canister 162 from the pump assembly 160.

In other arrangements, the projections 348 formed on the upper surface 349 of the canister 162 can be used to facilitate a single hand removal of the canister 162 from the pump assembly 160, but not to force the canister 162 away from the pump assembly 160 as described with respect to other arrangements above. The projections 348 formed on the upper surface 349 of the canister 162 can engage the first and second projections 350, 352 to hold the first and second projections 350, 352 in an open position to thereby permit a user to remove the canister 162 from the pump assembly 160 without requiring the user to hold the first and second projections 350, 352 in the open position. For example, as described above, pushing the canister release button 202 will cause the first and second projections 350, 352 of the actuator 262 to move over the sloping surface of the projections 348 formed on the upper surface 349 of the canister 162 until the first and second projections 350, 352 have moved past the projections 348. In this position, the orthogonal surfaces of the projections 348 on the canister 162 will prevent the first and second projections 350, 352 and, hence, the actuator 262, from moving back to the initial or locked position such that a user can then remove the canister 162 from the pump body 160 without having to continue pressing the canister release button 202. In this arrangement, a user can remove the canister 162 from the pump assembly 160 with a single hand operation (i.e., with one hand).

The canister 162 can be configured to include all of the disposable or serviceable items typically associated with the canister or use of a canister (including, for example and without limitation, the sealing ring or gasket for sealing the canister and the odour filter) as part of the removable canister component such that the sealing ring and the odour filter will be removed with the canister when the canister 162 is removed from the pump assembly 160. This can improve the efficiency and ease with which a new canister can be installed on a pump assembly 160 such that ‘between-patient’ servicing can be streamlined. In some arrangements, the ‘between-patient’ servicing can include the following steps or, in some arrangements, can include only the following steps: removing the used or partially filled canister 162, cleaning at least the external surfaces of the pump assembly 160 with a disinfecting cleaner, installing a new canister 162, and performing a self-test of the pump assembly 160 via the user interface of the pump assembly 160. Again, this can great simplify the servicing procedure and allow for simpler capital purchase and rental supply modes for some arrangements of the negative pressure wound treatment system 100.

In some arrangements, the canister 162 can include the canister body 346. The canister body 346 of some arrangements can include a blow-molded one piece design made from a clear polymer that can allow the exudate to be viewed, or a translucent polymer that permits a level of the exudate to be determined. The canister body 346 can be made from a naturally translucent material such as polypropylene or high density polyethylene, which are low cost and non-toxic.

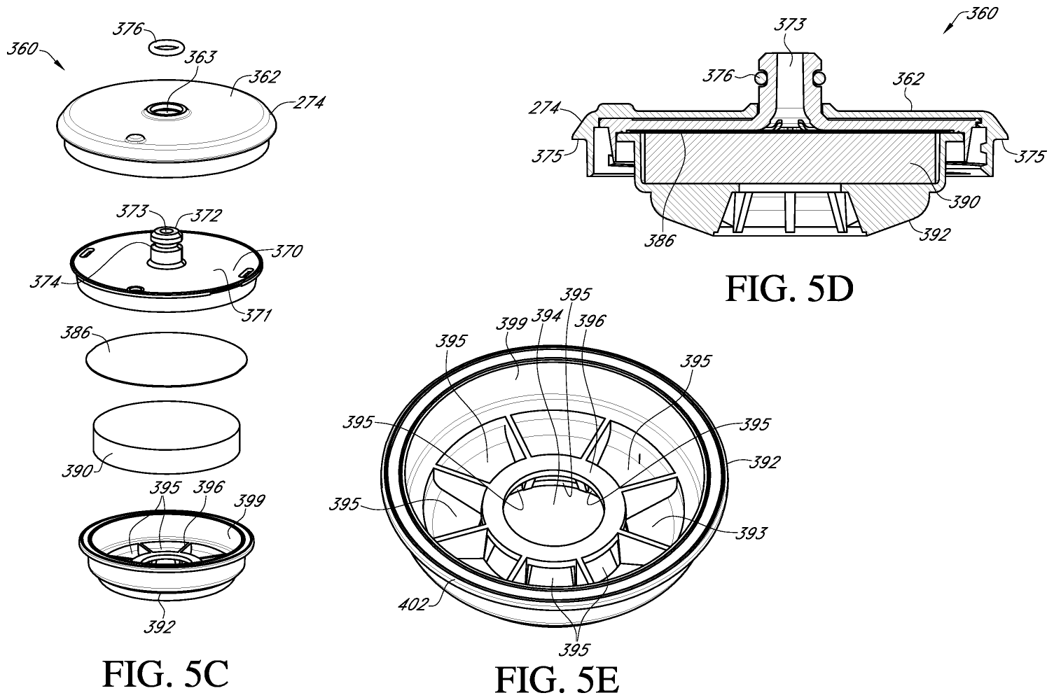

Some arrangements of the canister 162 can have a cap assembly 360 that can be configured to be removably coupled (for example and without limitation, threadedly coupled) with an opening of the canister body 346. FIGS. 5A-5E show an arrangement of the cap assembly 360 that can be included in any of the canisters 162 disclosed herein. With reference to FIGS. 5A-5E, some arrangements of the cap assembly 360 can include a cover 362 having the flange 274 (which can be an annular flange) and an opening 363 extending axially through a center portion of the first cap member 362.

The cap assembly 360 can also have a cap body 370 having a connector interface 372 projecting axially away from a first main surface 371 of the cap body 370. The connector interface 372 can have a generally cylindrical shape and an opening 373 extending axially therethrough. The opening 373 can be configured to provide a fluid passageway for air and/or other gases within the canister body 346 to pass and to exit the canister body 346 through. The connector interface 372 can also have an annular groove 374 configured to receive and support a sealing ring 376 therein. The sealing ring 376 can be a rubber O-ring or an O-ring from silicone or from another suitable material.

The cap assembly 360 can also have a hydrophobic filter 386 and an odor filter 390. The odor filter 390 can also be configured to filter out bacteria from the air flowing through the filter 390. The hydrophobic filter 386 can be used to prevent any liquids from escaping from the canister body 346 through the opening 373 in the cap body 370 and can be positioned on either or both sides of the odor filter 390. The odor filter 390 can include any suitable filter membrane or material, including carbon. For example and without limitation, some arrangements of the odor filter 390 can include compressed carbon.

Conventional negative pressure wound therapy pumps often get complaints for bad odor. Because odor filters are typically placed on the device exhaust, over time, odors have a tendency to build up within the internal tubing and the pump motor. The arrangements of this disclosure provide a filter (e.g., filter 390) at the canister 162 to prevent or at least inhibit the passage of any bacteria or other odor causing substances from exiting the canister 162 by preventing such bacteria and other odor causing substances from passing through the cap assembly 360 into the pump assembly 160. This arrangement also has the benefit of preventing the buildup of bacteria and other odor causing substances from contaminating the pump assembly 160, thereby permitting the reuse of the pump assembly 360 without requiring a substantial cleaning of the air passageways through the pump assembly 360.

In some arrangements, the filter 390 can include a carbon activated foam material. In some arrangements, the filter 390 can include a compressed carbon element as part of the filtration system in the canister. The carbon element can have various shapes and sizes depending on the canister. The carbon element could be the first part of the filtration system followed by a hydrophobic membrane. This would, in some arrangements, ensure that odor is the first thing that is filtered as air is pulled from the canister to the pump. As shown, this filtration system can be attached or form part of the canister cap assembly 360. Additionally, including the odor filter in the canister can eliminate the costly and more difficult task of replacing an odor filter within the inside of the pump assembly.

Different sizes and shapes for the carbon filter can be employed to increase its effectiveness for different canister shapes. Nevertheless, the premise of having a filtration system where air must go through a carbon disk and then a hydrophobic membrane still remains the same for each of these different sizes and shapes of the carbon filter element and the cap assembly 360. For any arrangements, a spacer may be placed between the carbon filter and the hydrophobic filter. This may be required to support the membrane and hold the carbon filter in the desired position.

The cap assembly 360 can also include a base cap support 392 that can be configured to provide a support surface for the filter 390 and/or other components of the cap assembly 360. The base cap support 392 can also have a shield or wall 393 configured to overlap or cover at least a portion of the filter 390 so as to inhibit or prevent liquid or exudate within the canister 346 from splashing onto at least a portion of the filter 390 and/or the hydrophobic filter 386. For example and without limitation, the shield 393 can overlap at least 40% of a surface area of a first main surface of the filter 390, or at least 50% of a surface area of the first main surface of the filter 390, or from at least 40% to at least 60% of a surface area of the first main surface of the filter 390.

The shield 393 can have an opening 394 therein that air and/or other gases can pass through as the air and/or other gases are being drawn through the cap assembly 360 when the pump is in operation. In some arrangements, the shield 393 can block a majority of a surface, or 60% or approximately 60% of a surface of the filter 390 from exposure to exudate within the canister 346. In other words, in some arrangements, the opening 394 extending through the base cap support 392 can be reduced by 60% or approximately 60% or more with the shield 393 as compared to an opening through the base cap support 392 that does not include the shield 393. In some arrangements, the opening 394 extending through the base cap support 392 can be reduced by from 40% (or approximately 40% or less) to 80% (or approximately 80% or more), or from 50% (or approximately 50%) to 70% (or approximately 70%) as compared to an opening through the base cap support 392 that does not include the shield 393.

The base cap support 392 can have a bottom support or standoff 396 that can support the filter 390. One or more fluid passageways 395 can be formed through the bottom support or standoff 396. The passageways can be in communication with a recess 399 formed in the base cap support 392 so that air that passes through the passageways 395 can also pass through or fill the recess 399. In some arrangements, the recess 399 can be sized and configured to receive and support the filter membrane 390 therein. The recess 399 can be configured such that all air or gas or substantially all air or gas coming from the canister body 346 must pass through the filter 390 before passing through the opening 373 in the cap assembly 360. The base cap support 392 can also have an annular flange or lip 402 thereon.

Some arrangements of the base cap support 392 can also include one or more projections 400 extending axially away from an upper surface of the cap assembly 360 so that, in operation, the one or more projections 400 (two being shown) extend into an inside space in the canister body 346.

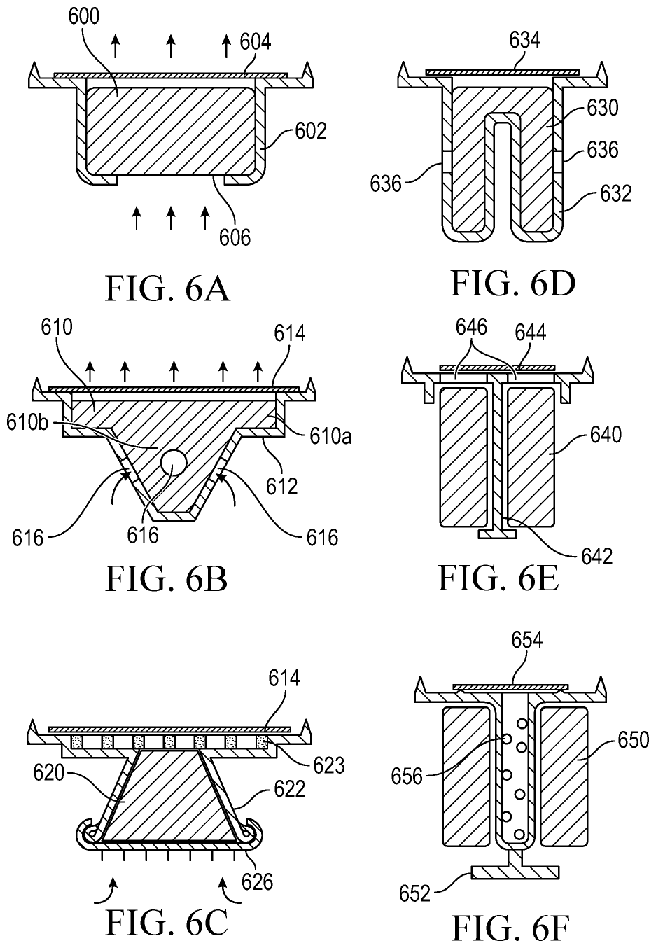

FIGS. 6A-6F show other arrangements of a filter 600 and filter support 602 that can be used with any arrangements of the negative pressure wound treatment system 100 disclosed herein, in place of or in addition to the other filters described herein for the other arrangements of the negative pressure wound treatment system 100. For example, as shown in FIG. 6A, some arrangements of the filter 600 and filter support 602 shown therein can filter odors, bacteria, and other substances or materials from the air being drawn from the collection canister (such as canister 162). A hydrophobic filter 604 can be positioned on one or both sides of the filter 600. Air can flow through a first opening 606 in the filter support 602, then be forced to pass through the filter 600 and hydrophobic filter 604 before passing into the pump assembly (such as pump assembly 160). In the variation shown in FIG. 6B, air can be forced to pass through a plurality of openings 616 in the filter support 612 before passing through the filter 610 and the hydrophobic filter 614.

In the variation of the filter 610 shown in FIG. 6B, the filter 610 can have a disk shaped portion 610a and a tapering or conically shaped portion 610b that extends downward toward an inside of the canister with the filter becoming increasingly narrow as the filter extends away from the disk shaped portion of the filter, for example. The filter support 612 can have a shape the complements the shape of the filter 610. The openings 616 can pass through a conically shaped or tapered portion of the filter support 612. This arrangement can increase the flow path of the air through the filter 610 to increase a filtration of the air exiting the canister, and can also increase the surface area of the covering over the filter 610 to reduce an amount of the filter 610 that can be exposed to splashing or sloshing exudate within the canister.

The variation of the filter 620 shown in FIG. 6C can have a tapering shape that increases in size toward a bottom end of the filter 620 and is the narrowest at an upper end thereof. The hydrophobic filter 624 can be spaced apart from the filter 620 by a spacer member 623. The filter support 622 can have a shape that complements a shape of the filter 620, the spacer member 623, and the hydrophobic filter 624. Openings 626 in a bottom surface of the filter support 622 can provide a fluid passageway for air through the filter 620. FIGS. 6D-6F show additional variations of shapes of the filters 630, 640, 650 and the filter supports 632, 642, 652 that can be used in any of the arrangements of the negative pressure wound treatment systems 100 disclosed herein. Hydrophobic filters 634, 644, 654 can be positioned over or adjacent to any of the filters 630, 640, 650, and one or more openings 636, 646, 656 can permit air flow through the filter supports 632, 642, 652. In some arrangements, the negative pressure wound treatment system 100 can have an odor filter that filters the exhaust flow that exits from the pump module 248. The odor filter that filters the exhaust flow that exits from the pump module 248 can be positioned inside of the housing 210 or external to the housing 210, which can provide easier access for servicing and/or replacing the odor filter (i.e., if located external to the housing 210). In some arrangements, the filter can be attached to the top of the canister 162.

In some arrangements, the operation of such a filter can be described as follows. The pump can pull a negative pressure onto the canister contents via a first port, thus drawing air and exudate from the dressing. Exudate can be separated in the canister, and air can be pulled through the pump unit, and exhausted to a second port. A carbon filter can be attached to an exterior surface of the canister and, when the canister is correctly attached to the device, the filter is pushed up against the second port. Exhaust air can be forced through the filter (which can be a carbon filter or any other suitable odor and/or bacteria type filter). The filter can remove the bacteria byproducts before venting the exhaust air to atmosphere.

Arrangement of the filters disclosed herein can use simple and low cost filter technology—for example and without limitation, a foam with carbon impregnation.

Locating the filter on or in the canister can guarantee a filter change when the canister is changed. Additionally, the filter can provide exhaust muffling. In some arrangements, the filter can be a wet-side filter located inside the canister. Additionally, in some arrangements, one or more additives can be added to the canister to reduce bacteria count or growth. Further, in some arrangements, a dry filter can be placed in the canister cap, but in a separate compartment accessible only by the pump unit exhaust, not at the pump inlet. This can, in some arrangements, include two ports on the canister cap—an inlet port and an outlet port.

Traditional NPWT pumps generate noise when in operation. The noise tends to come from the mechanical movement of components in the pump (generally the pump head) as well as form the air that is pulled and exhausted by the pump. This noise can be a nuisance to the patient, and can be directly linked to therapy outcomes since patients may have a tendency to turn the pump off to stop the noise. These pauses in therapy delay the wound healing process.

Some arrangements of the pump assembly 160 disclosed herein can include inline filters connected to tubing inside the pump enclosure as well as baffle boxes to mitigate the noise from the pump motor. Some arrangements of the pump assembly 160 can include a filter membrane on an exhaust port on the pump assembly 160. The filter membrane can include Porex PTFE membrane vents, Pall Versapor (for example and without limitation, a Pall Versapor 1200 filter) vents, or any sheet type membrane that can be attached to the housing of the pump assembly 160 at the exhaust port. In some arrangements, the filter can have the shape of a disk and can be adhered to the housing of the pump assembly 160. In some arrangements, the filter can be encased in a housing, a cover, or a clamping lid that can be attached to an outside surface of the housing of the pump assembly 160. The filter can also include a paper filter sheet. Additionally, some arrangements of the filter can be configured to create an impermeable barrier that prevents any fluid from entering the enclosure if the device is placed upside down or tipped. Additionally, such filters can also be configured to provide additional filtration against and bad odors exhausting from the pump motor.

In some arrangements, the tubing 142 that is connected with the canister 162 can have a length that is sufficient to connect with a dressing or port that is at least two feet, or at least three feet, or between one foot and three feet or more away from the canister 162. The tubing 142 can be coupled with a connector 440 that extends away from a surface of the canister 162. The connector 440 can be in fluid communication with an interior space within the canister body 346, and can be configured to couple with an end portion of the tubing 142. All or a portion of the tubing 142 can wrap around the canister 162 when all or a portion of the tubing 142 is not needed.

Some arrangements of the negative pressure wound treatment system 100 can be configured to support the tubing 142 on or in the pump assembly 160 and/or the canister 162 so as to manage the tubing 142. Tubing (such as tubing 142) has been reported by patients to be intrusive during everyday life. As wounds can be on different parts of the body, some arrangements of the negative pressure wound treatment system 100 can be provided with a length of tubing 142 to accommodate the farthest away positioned wounds. Configuring the negative pressure wound treatment system 100 to manage any excess tubing can be beneficial in terms of preventing tripping hazards, preventing tipping hazards, and maintaining a more orderly the negative pressure wound treatment system 100. Therefore, some arrangements of the negative pressure wound treatment system 100 disclosed herein can have one or more clips that removably or nonremovably attach to the exudate tubing 142 and can be used to secure the excess tubing to the negative pressure wound treatment system 100. In some arrangements, the tubing can be wrapped around the pump assembly 160 and/or the canister 162, minimizing the profile of the tubing and allowing the user to control a length of the tubing that extends away from the negative pressure wound treatment system 100.

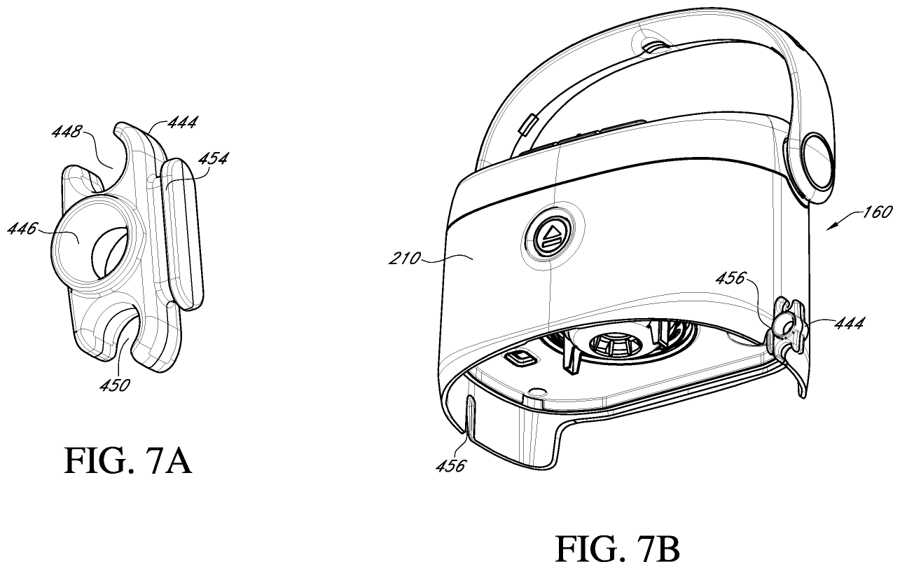

With reference to FIGS. 7A-7B, in some arrangements, one or more tubing supports 444 (also referred to as clips) can be coupled with or attached to the housing 210 of the pump assembly 160 (as shown) or to the canister body 346 (not shown) and can be configured to selectively support the tubing 142. Some arrangements of the tubing supports 444 can be removably attached to or coupled with the canister body 346. Additionally, the tubing supports 444 can have an enclosed opening 446 through which the tubing 142 can pass, a first open support 448 to which a portion of the tubing can be removably secured or retained, and/or an optional second open support 450 to which a portion of the tubing can be removably secured or retained. The enclosed opening 446 can be used to nonremovably attach the tubing 146 to the tubing support 444. The tubing supports 444 can also have a retaining portion 454 that can be used to secure the tubing support 444 to a receiving portion 456 in the housing 210 of the pump assembly 160. The tubing supports 444 can be slid into and out of the slots 456 to selectively secure the tubing supports 444 to the pump assembly 160 (as shown) or to the canister body 346 (not shown).

In some implementations, the tubing support 444 can be secured to the housing 210 using a slot or other attachment element on the housing 210 or the canister body 346. In this implementation, the tubing could then be wrapped around the housing 210 and the tubing support 444 would secure the additional loops of the tubing that go around the housing 210 to the housing 210. The tubing support 444 can also be configured to be slid onto the tubing (e.g., the tubing could be passed through the enclosed opening 446 in the tubing support 444) so that the tubing support is at approximately a midpoint position on the tubing. Then, the tubing can be formed into a coil that can be removably secured to the tubing support 444.

Some arrangements of the negative pressure wound treatment system 100 can have two or more tubing supports 444, one on each of two side portions of the negative pressure wound treatment system 100, as shown. In other arrangements, the negative pressure wound treatment system 100 can have only one tubing support 444, or three or more tubing supports 444, depending on the length of the tubing 142. Additionally, the tubing supports 444 can be positioned on any desired portion of the pump assembly 160 and/or the canister 162.

The tubing supports 444 can have any desired shape or feature. For example, some implementations of the tubing supports 444 can be configured to have a strap around the openings or retaining portions to retain the tubing 142 to the tubing support 444. In other arrangements, the tubing 142 can be supported in an internal space or cavity within the pump assembly 160 and/or the canister 162 in which the tubing can be coiled up. In this arrangement, the user can pull a required or desired length of the tubing from the internal space and then can actuate a locking mechanism to secure the remainder of the tubing in the internal space. In other arrangements, the tubing can be supported in a pouch or cavity that is attached to an outside of the pump assembly 160 and/or the canister 162. In some arrangements, the tubing can be fused together such that a user must separate out the portion of the tubing that the user intends to use, with the remaining portion of the tubing being secured to or retained by the negative pressure wound treatment system 100.

In some arrangements, the pump assembly 160 can be configured such that a center of gravity is much lower than in conventional pump assemblies. This can improve the stability of the pump assembly 160 and the negative pressure wound treatment system 100 and reduce the instances of inadvertent tipping over of the negative pressure wound treatment system 100. For example and without limitation, the center of gravity can be at approximately 40% of the total height of the pump assembly 160 (not including the handle 208), or from 35% (or approximately 35%) of the total height of the pump assembly 160 (not including the handle 208) to 50% (or approximately 50%) of the total height of the pump assembly 160 (not including the handle 208). Additionally, as shown, the buttons and other inputs of the interface panel 170 can be on a top surface of the pump assembly 160 such that an input force applied to the pump assembly 160 when a user provides a physical input to the pump is in a mostly downward direction. This can also reduce the chances of the pump assembly 160 being inadvertently overturned when a user provides an input into the pump assembly 160.

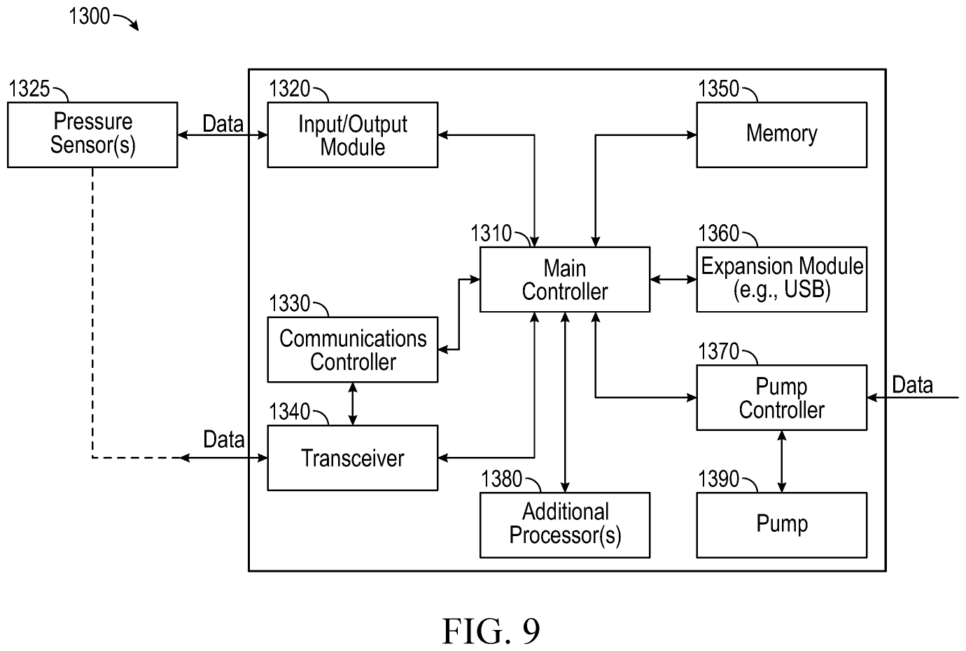

FIG. 9 illustrates a schematic of a control system 1300 that can be employed in any of the wound therapy devices described herein, such as in the wound therapy device 110′ and/or wound therapy device 110. The control system 1300 can be similar to the electronic assembly described herein. Electrical components can operate to accept user input, provide output to the user, operate the pressure source, provide connectivity, and so on. A first processor (such as, a main controller 1310) can be responsible for user activity, and a second processor (such as, a pump controller 1370) can be responsible for controlling another device, such as a pump 1390.

An input/output (I/O) module 1320 can be used to control an input and/or output to another component or device, such as the pump 1390, one or more sensors (for example, one or more pressure sensors 1325 configured to monitor pressure in one or more locations of the fluid flow path), or the like. For example, the I/O module can receive data from one or more sensors through one or more ports, such as serial (for example, I2C), parallel, hybrid ports, and the like. Any of the pressure sensors can be part of the wound therapy device or the canister. In some cases, any of the pressure sensors 1325 can be remote to the wound therapy device, such as positioned at or near the wound (for example, in the dressing or the conduit connecting the dressing to the wound therapy device). In such implementations, any of the remote pressure sensors can communicate with the I/O module over a wired connection or with one or more transceivers 1340 over a wireless connection.

The main controller 1310 can receive data from and provide data to one or more expansion modules 1360, such as one or more USB ports, SD ports, Compact Disc (CD) drives, DVD drives, FireWire ports, Thunderbolt ports, PCI Express ports, and the like. The main controller 1310, along with other controllers or processors, can store data in memory 1350 (such as one or more memory modules), which can be internal or external to the main controller 1310. Any suitable type of memory can be used, including volatile or non-volatile memory, such as RAM, ROM, magnetic memory, solid-state memory, Magnetoresistive random-access memory (MRAM), and the like.

The main controller 1310 can be a general purpose controller, such as a low-power processor or an application specific processor. The main controller 1310 can be configured as a “central” processor in the electronic architecture of the control system 1300, and the main controller 1310 can coordinate the activity of other processors, such as the pump controller 1370, one or more communications controllers 1330, and one or more additional processors 1380. The main controller 1310 can run a suitable operating system, such as a Linux, Windows CE, VxWorks, etc.

The pump controller 1370 can control the operation of a pump 1390, which can generate negative or reduced pressure. The pump 1390 can be a suitable pump, such as a diaphragm pump, peristaltic pump, rotary pump, rotary vane pump, scroll pump, screw pump, liquid ring pump, diaphragm pump operated by a piezoelectric transducer, voice coil pump, and the like. The pump controller 1370 can measure pressure in a fluid flow path, using data received from one or more pressure sensors 1325, calculate the rate of fluid flow, and control the pump. The pump controller 1370 can control the pump actuator (such as, a motor) so that a desired level of negative pressure is achieved in the wound 104. The desired level of negative pressure can be pressure set or selected by the user. The pump controller 1370 can control the pump (for example, pump motor) using pulse-width modulation (PWM) or pulsed control. A control signal for driving the pump can be a 0-100% duty cycle PWM signal. The pump controller 1370 can perform flow rate calculations and detect alarms. The pump controller 1370 can communicate information to the main controller 1310. The pump controller 1370 can be a low-power processor.

Any of the one or more communications controllers 1330 can provide connectivity (such as, a wired or wireless connection 1332). The one or more communications controllers 1330 can utilize one or more transceivers 1340 for sending and receiving data. The one or more transceivers 1340 can include one or more antennas, optical sensors, optical transmitters, vibration motors or transducers, vibration sensors, acoustic sensors, ultrasound sensors, or the like. Any of the one or more transceivers 340 can function as a communications controller. In such case, the one or more communications controllers 330 can be omitted. Any of the one or more transceivers 340 can be connected to one or more antennas that facilitate wireless communication. The one or more communications controllers 1330 can provide one or more of the following types of connections: Global Positioning System (GPS), cellular connectivity (for example, 2G, 3G, LTE, 4G, 5G, or the like), NFC, Bluetooth connectivity (or BLE), radio frequency identification (RFID), wireless local area network (WLAN), wireless personal area network (WPAN), WiFi connectivity, Internet connectivity, optical connectivity (for example, using infrared light, barcodes, such as QR codes, etc.), acoustic connectivity, ultrasound connectivity, or the like. Connectivity can be used for various activities, such as pump assembly location tracking, asset tracking, compliance monitoring, remote selection, uploading of logs, alarms, and other operational data, and adjustment of therapy settings, upgrading of software or firmware, pairing, and the like.

Any of the one or more communications controllers 1330 can provide dual GPS/cellular functionality. Cellular functionality can, for example, be 3G, 4G, or 5G functionality. The one or more communications controllers 1330 can communicate information to the main controller 1310. Any of the one or more communications controllers 1330 can include internal memory or can utilize memory 1350. Any of the one or more communications controllers 1330 can be a low-power processor.

The control system 1300 can store data, such as GPS data, therapy data, device data, and event data. This data can be stored, for example, in memory 1350. This data can include patient data collected by one or more sensors. The control system 1300 can track and log therapy and other operational data. Such data can be stored, for example, in the memory 1350.

Using the connectivity provided by the one or more communications controllers 1330, the control system 1300 can upload any of the data stored, maintained, or tracked by the control system 1300 to a remote computing device, such as the device 1334. The control system 1300 can also download various operational data, such as therapy selection and parameters, firmware and software patches and upgrades, and the like (for example, via the connection to the device 1334). The one or more additional processors 1380, such as processor for controlling one or more user interfaces (such as, one or more displays), can be utilized. In some cases, any of the illustrated or described components of the control system 1300 can be omitted depending on an arrangement of a wound monitoring or treatment system in which the control system 1300 is used.

Any of the negative pressure wound therapy devices described herein can include one or more features disclosed in U.S. Pat. No. 9,737,649 or U.S. Patent Publication No. 2017/0216501, each of which is incorporated by reference in its entirety.

### Multiple Dressing Negative Wound Therapy

FIG. 10 illustrates another negative pressure wound treatment system 1400. The system 1400 can include a wound therapy device capable of supplying negative pressure to the wound site or sites, such as wound therapy device 110. The wound therapy device 110 can be in fluidic communication with one or more wound dressings 1406a, 1406b (collectively referred to as 1406) so as to supply negative pressure to one or more wounds, such as the wounds 104a and 104b. The fluidic connection between a wound dressing 1406 and a wound therapy device 110 can be referred to as a fluid flow path (e.g., the path through which fluid aspirated from a wound via negative pressure flows). For instance, a first fluid flow path can include components providing fluidic connection from the wound therapy device 110 to the first wound dressing 1406a. As a non-limiting example, the first fluid flow path can include the path from the wound dressing 1406a to the wound therapy device 110 or the path from the first wound dressing 1406a to an inlet 1446 of a branching attachment (or connector) 1444 in fluidic connection with the wound therapy device 110. Similarly, a second fluid flow path can include components providing fluidic connection from the wound therapy device 110 to the second wound dressing 1406b.

The system 1400 can be similar to the system 100 with the exception that multiple wounds 104a and 140b are being treated by the system 1400. The system 1400 can include any one or more of the components of the system 100, which are illustrated in FIG. 4 with appended letter “a” or “b” to distinguish between the first and second wounds (such as, the wounds 104a and 104b, the covers 106a and 106b). As illustrated, the system 1400 can include a plurality of wound dressings 1406a, 1406b (and corresponding fluid flow paths) in fluidic communication with the wound therapy device 110 via a plurality of suction adapters, such as the adapter 108. The suction adapters can include any one or more of the components of the adapter 108, which are illustrated in FIG. 4 with appended letter “a” or “b” to distinguish between the first and second wounds (such as, the bridge portions 130a and 130b, the connectors 134a and 134b, and the caps 140a and 140b).

Without limitation, the suction adapters for the systems 1400a and 1400b can include a controlled leak channel fluidically separate from a suction channel. Each wound dressing and fluid flow path can include a variety of features or elements which match or are similar to features or elements of another wound dressing or fluid flow path within the system. For ease of reference, one or more corresponding features or elements can be collectively referred to using a reference number without a corresponding letter. For example, wound dressing 1406a and wound dressing 1406b can be collectively referred to as wound dressing 1406. However, it should be noted that, in some arrangements, elements which have been collectively referred to are not identical and can have different features or attributes.

In some arrangements, the dressings 1406a, 1406b can be placed over an aperture or opening formed in each of respective drapes or wound covers 106a, 106b that are placed over a suitably-prepared wounds 1430a, 1430b, which can in some cases be filled with a wound packing material such as foam or gauze. The wound therapy device 110 can be fluidically coupled via the tube 142 with the inlet 1446 of the connector 1444. The connector 1444 can be fluidically coupled via branches 1445a, 1445b and tubes or conduits 1442a, 1442b with the connectors 134a, 134b, which can be fluidically coupled with the tubes or conduits 130a, 130b. The tubes or conduits 130a, 130b can be fluidically coupled with the dressings 1406a, 1406b. Once all conduits and dressing components are coupled and operably positioned, the wound therapy device 110 can be activated, thereby supplying negative pressure via the fluid flow paths to the wounds 1430a, 1430b. Application of negative pressure can be applied until a desired level of healing of the wounds 1430 is achieved. Although two wounds and wound dressing are illustrated in FIG. 4, some implementations of the wound therapy device 110 can provide treatment to a single wound (for instance, by closing the unused branch 1445a or 1445b of the connector 1444) or to more than two wounds (for instance, by adding branches to the connector 1444).

In any arrangements disclosed herein, the inlet manifold branching attachment 1444 or the conduit can include one or more valves, clamps, caps, air leaks, or other flow regulator mechanisms which can be configured to admit fluid into a fluid flow path or, alternatively, block or restrict flow or passage of fluid through a fluid flow path. In some arrangements, valves, air leaks, or other flow regulation mechanisms in the inlet manifold branching attachment 1444 can be opened or closed electronically. For instance, a controller of the wound therapy device 110 can communicate with the valves, air leaks, etc. to open or close each one individually or as a unit. This communication can be wired or wireless.

In some arrangements, the system 1400 can apply negative pressure to one or more wounds. The level of negative pressure at one or more of the wounds (for example, under one or more wound dressings) can be sufficiently close to the negative pressure level at the source of negative pressure. For example, an acceptable level of pressure maintained at the wound can be within ±1 mmHg, ±5 mmHg, ±10 mmHg, ±25 mmHg, and the like of the negative pressure set point. In some arrangements, this pressure can be maintained at this level within 95% (or another suitable percentage) of the time that the system 1400 has negative pressure applied to it. In some arrangements, acceptable pressure levels can include pressure ranges between −40 to −120 mmHg. However, other pressure levels can be used as described herein.

One or more air leaks such as the air leaks in one or more of the fluid flow paths can be utilized to determine one or more operating conditions within the system. For example, an air leak can be a controlled air leak that can admit a relatively constant air, gas, or other fluid flow into a fluid flow path. In some arrangements, the flow into the fluid flow path from an air leak can be configured and/or controlled to not appreciably increase as additional negative pressure is applied to the system. However, the presence of an air leak in the system can maintain substantially constant baseline flow through the system when steady state has been achieved (for example, when the negative pressure set point has been reached). In turn, presence of the air leak can require the negative pressure source to work harder to maintain the desired level of negative pressure at the wound(s). Accordingly, the system can determine the presence of one or more operating conditions (such as a blockage, leakage, canister full, misalignment of the suction adapter and the like) by monitoring the flow through the fluid flow path(s), which can be measured directly or indirectly based on, for example, monitoring an activity of the negative pressure source.

In some arrangements, each fluid flow path can include an air leak and each air leak of a respective fluid flow path can admit a different flow rate of air, gas, or other fluid into the system. In other words, each air leak of the system can have a different leak rate. For example, the leak rate of an air leak can be based at least in part on the size or shape of the air leak, whether the air leak includes a filter, the size or porous level or a filter, a level of occlusion of the air leak or the filter, and the like. The fluid admitted into a fluid flow path can increase the flow rate of that fluid flow path.

Accordingly, each fluid flow path of the system 1400 can have a different flow rate. The total flow rate (TFR) of the system 1400 (e.g., the aggregation of the flow to each of the wound dressings) can be monitored, calculated, or determined and then used to determine an operating condition of the system 1400. Operating conditions can, for instance, include a “no flow” condition (e.g., all of the flow paths are blocked), a blockage condition of one or more flow paths (e.g., a blockage condition exists in a first fluid flow path, a blockage condition exists a second fluid flow path, etc.), a canister full condition, normal operation (e.g., no blockages are present in any of the fluid flow paths), and the like.

The system 1400 can include one or more features disclosed in U.S. Patent Publication No. 2020/0069850 or International Publication No. WO2018/167199, each of which is incorporated by reference in its entirety.

In some arrangements, the system 1400 can be capable of providing an indication, such as an alarm, to communicate to the user an operating status of the system 1400 based on a comparison of the determined total flow rate and one or more flow thresholds. In some arrangements, the flow thresholds corresponding to operating conditions of the system 1400 can be pre-determined. In some arrangements, the flow thresholds can be based at least in part on dynamic measurements or calculations of the system 1400, such as a flow rate or pressure, during a particular mode of the system (e.g., a calibration mode).

### Handle

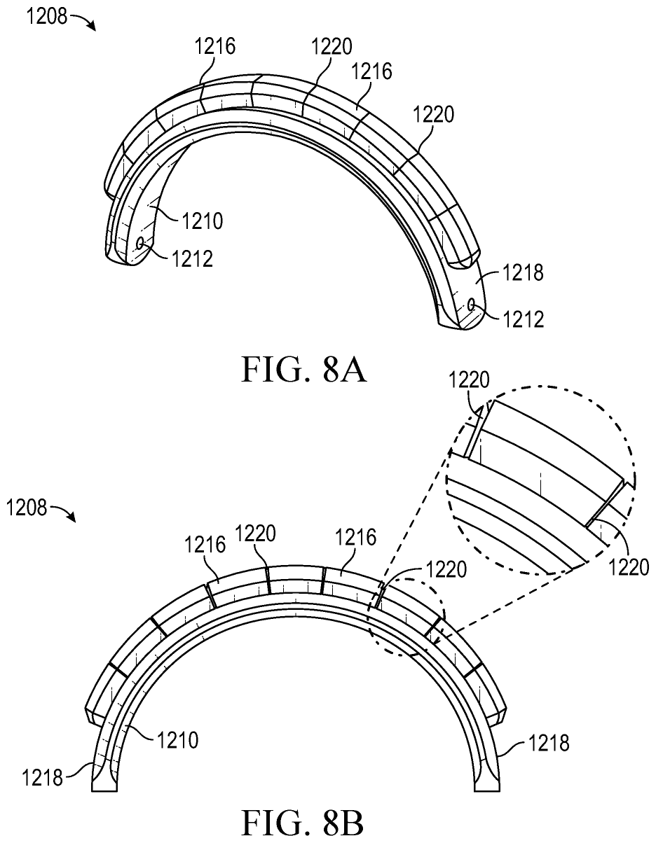

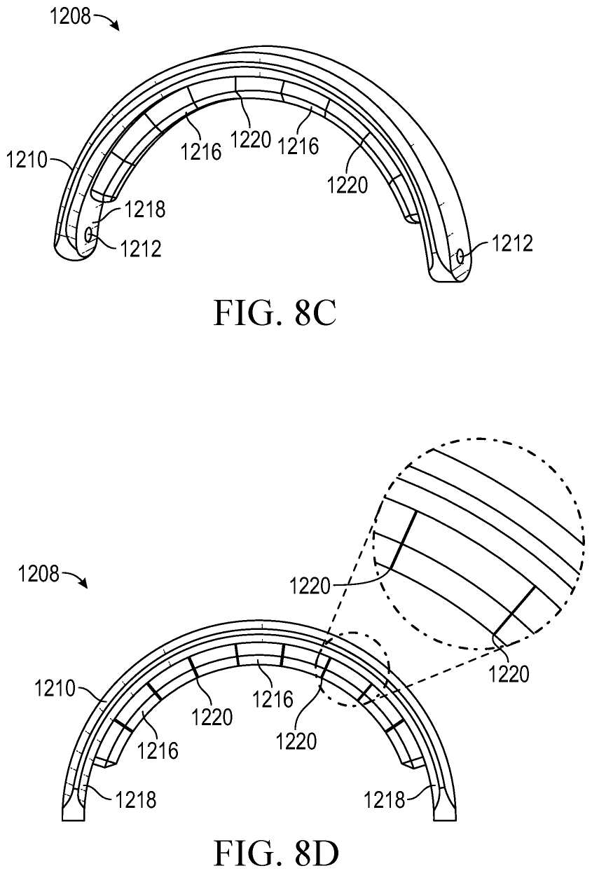

FIGS. 8A-8D illustrate another arrangement of handle 1208 that can be used with any implementations of the pump device (including, for example and without limitation, any arrangements of the pump device 160) disclosed herein. In some arrangements, the handle 1208 can be configured to be selectively changed between at least a first state and a second state. Some arrangements of the handle 1208 can be more flexible in the first state than in the second state and/or be more rigid or have a more solid feel in the second state than in the second state. In some arrangements, the handle 1208 can be configured to be changeable between the first state in the second state by flipping the handle 1208 over. For example, with reference to FIGS. 8A-8B, an implementation of the handle 1208 is shown in the first state in which the handle is more flexible. The same handle 1208 is shown in FIGS. 8C-8D in the second state, in which the handle is more rigid or has a more solid feel.

With reference to FIGS. 8A-8D, the handle 1208 can have a base portion 1210 having a first opening 1212 and a second opening 1212 through the end portions of the base portion 1210. The first and second openings 1212 can be used to couple the handle 1208 to the pump device using any suitable fasteners. However, in other arrangements, the handle 1208 can be coupled with the pump device using any suitable features, including reversible locking features similar to a zip tie, a ratchet mechanism, or other quick release connectors. For example, the pump device can have a depressive a feature that can be used to selectively release an end portion of the handle 1208 so that the handle can be removed and changed from the first day to the second state, or can be exchanged for a second handle that is either more rigid or more flexible. As a further example, some arrangements of the pump assembly 160 can have a have a lobe on the pump housing 210 that corresponds to a complementary feature in the handle 208 so that the handle 208 can be detached and/or attached only when the handle 208 is in the fully forward position. When the handle 208 is pulled upright or extended to the fully rear position, the lobe can be configured to engage with a ring inside the handle attachment boss that keeps the handle 208 coupled with the lobe, while permitting the handle 208 to freely rotate relative to the housing 210.

The handle 1208 can also have a plurality of compression elements 1216 projecting away from a first surface 1218 of the base portion 1210. When the handle 1208 is in the first or flexible state, as shown in FIG. 8A, the first surface 1218 can extend or faced generally outwardly and the compression elements 1216 will also face generally outwardly (which can be radially outwardly, in some arrangements). The compression elements 1216 can have a generally trapezoidal or tapered shape in some arrangements and can have a space 1220 between each of the compression elements 1216, at least when the handle 1208 is in the first or flexible state. When the compression elements 1216 are facing outwardly, as in the first state as shown in FIG. 8A, the curvature of the base portion 1210 can cause the spaces 1220 to expand or open up so that end portions of the compression elements 1216 are not in contact with one another. When the compression elements 1216 are facing inwardly, as in the second state as shown in FIG. 8C, the curvature of the base portion 1210 can cause the end portions of the compression elements 1216 to eliminate the space between the compression elements 1216 and contact one another and/or compress against one another to thereby cause the compression elements 1216 to increase the rigidity or stiffness of the handle 1208 and inhibit the flexibility of the handle 1208.

In other arrangements, the base portion 1210 can be made from a stretchable fabric with compression elements (also referred to herein as studs) each having a trapezoidal or suitable shape projecting away from one of the main surfaces of the base portion, preferably wherein the This would give more flexibility and strap like feel in the flexible orientation but retain the rigidity in the orientation where the trapezoidal studs meet. In any arrangements disclosed herein, the compression elements can have any desired shape and are not limited to trapezoidal shapes. For example and without limitation, the compression elements 1216 can have any suitable or desired shape that has a tapered face between each element that allows the handle to be flexible when there is space between each of the compression elements 1216 and more rigid when each of the compression elements 1216 are in contact with the adjacent compression elements 1216.

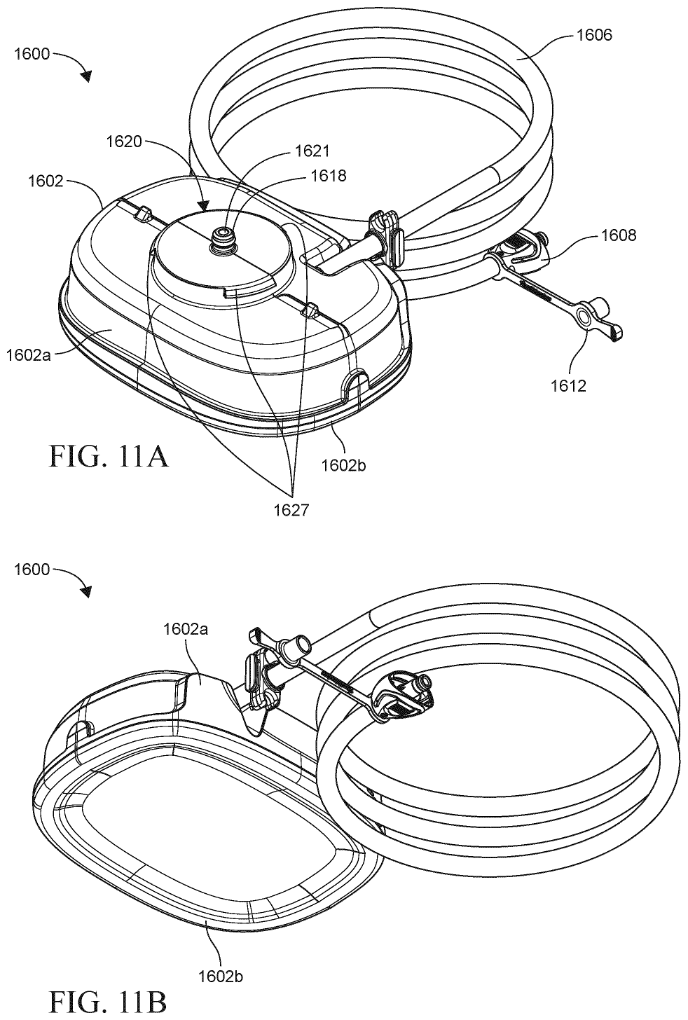

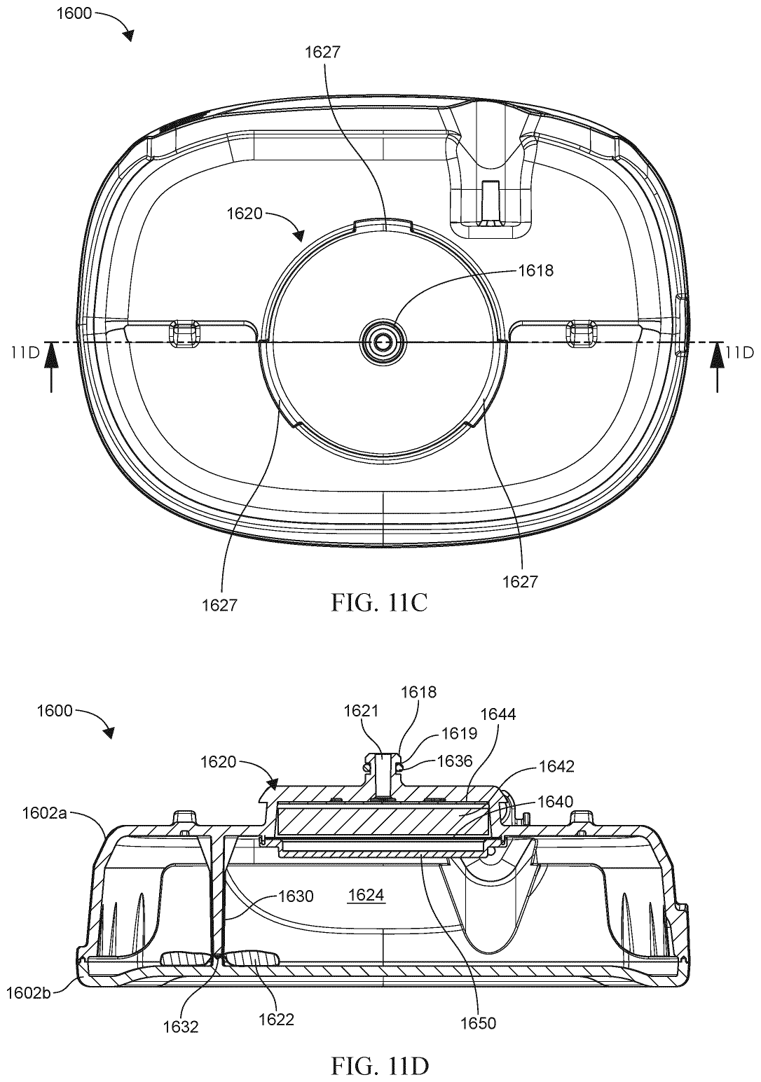

FIGS. 11A-11D illustrate an arrangement of a canister assembly 1600 that can be used with any of the pump assembly arrangements disclosed herein, including any arrangements of the pump assembly 160 disclosed herein. FIG. 11D is a section view of the canister assembly 1600 taken through line 11D-11D shown in FIG. 11C. Any arrangements of the canister assembly 1600 disclosed herein can have any of the components, features, or other details of any other canister assembly arrangements disclosed herein, including without limitation any of the arrangements of the canister 162 described above, in any combination with any of the components, features, or details of the canister assembly 1600 disclosed below. Similarly, any components, features, or other details of any of the other canister or canister assembly arrangements disclosed herein can have any of the components, features, or other details of any arrangements of the canister assembly 1600 disclosed herein in any combination with any of the components, features, or details of the canister or canister assembly.

In any arrangements disclosed herein, the canister assembly 1600 can have a canister body 1602, a conduit or tubing 1606 having a clip 1607 and a connector 1608 at a distal end thereof, a connector interface 1618, and a filter assembly 1620 housed within the canister body 1600. The canister body 1602 can have one or more, or a plurality of flanges 1627 (three being shown) configured to engage with or be engaged by latches, projections, or other selectively or nonselectively securable tabs or other features of the pump assembly (such as, without limitation, the latches 304, 306 of the locking arms 266, 268 and/or the latch 282 described above). The canister body 1600 can have a first or upper portion 1602a and a second or lower portion 1602b and can be any desired size, including 300 mL or approximately 300 mL, or from 200 mL or approximately 200 mL to 400 mL or approximately 400 mL. The first portion 1602a can be coupled with the second portion 1602b. For example and without limitation, the first portion 1602a can be welded with, adhered to, or otherwise secured with the second portion 1602b. In other arrangements, the first portion 1602a can be integrally formed with the second portion 1602b. The components of the canister body 1602 can be injection molded or formed by any other suitable method. A sealing ring 1636 can be positioned around an outside surface of the connector interface 1618 (e.g., within an annular groove 1619 formed in the connector interface 1618. In some arrangements, the connector interface 1618 can be formed integrally with the first body portion 1602a. A removable cap 1612 for the tubing connector 1608 can be tethered to the tubing 1606 near the tubing connector 1608.

In any arrangements disclosed herein, the canister assembly 1600 can also have a gelling agent 1622 (which can be in a package or bag) positioned within the interior space 1624 of the canister body 1602 configured to increase a thickness or viscosity of the liquid, which can include wound exudate, within the interior space 1624. In some arrangements, the gelling agent 1622 can be secured between a first projection 1630 extending away from an inside wall of the first portion 1602a toward the second portion 1602b and a second projection 1632 extending away from an inside wall of the second portion 1602b toward the first portion 1602a. For example, the gelling agent 1622 can be pinched between the first and second projections 1630, 1632. In this arrangement, the first and second projections 1630, 1632 can be used to prevent the gelling agent 1622 from moving around the interior space 1624 of the canister body 1602.

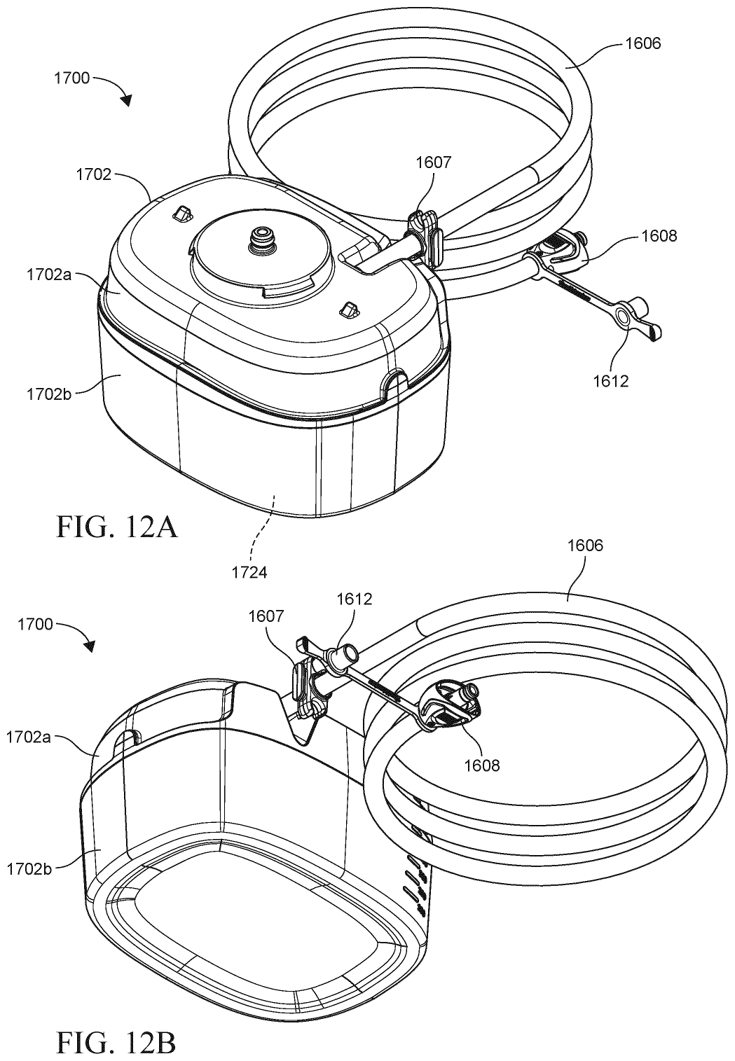

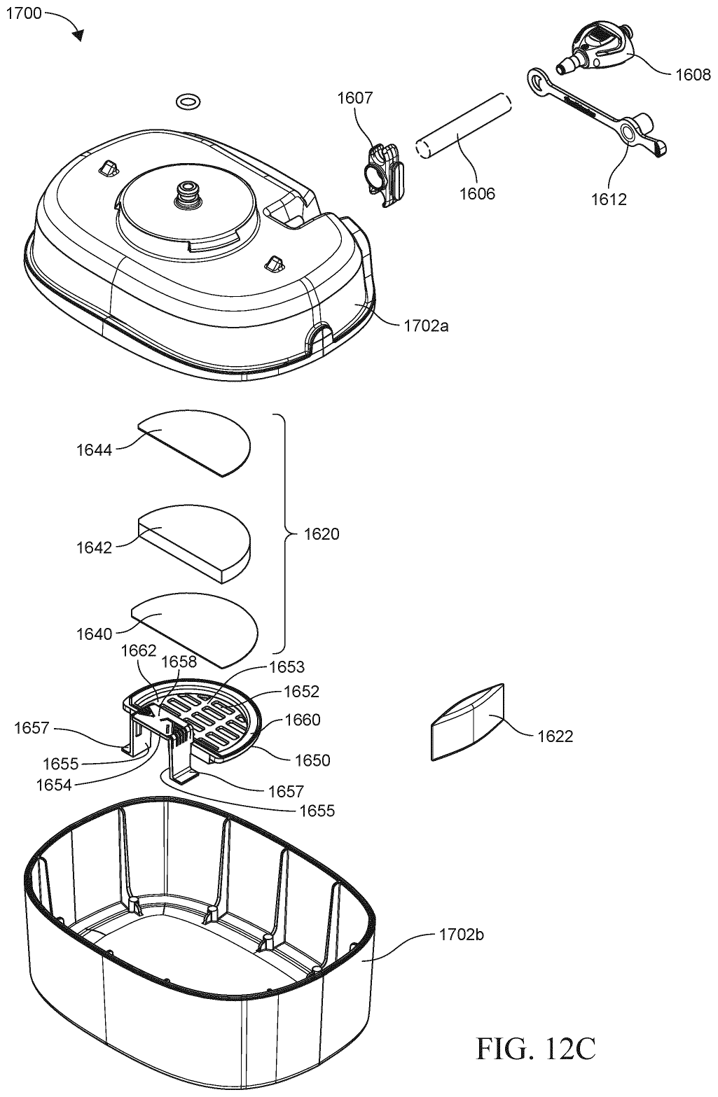

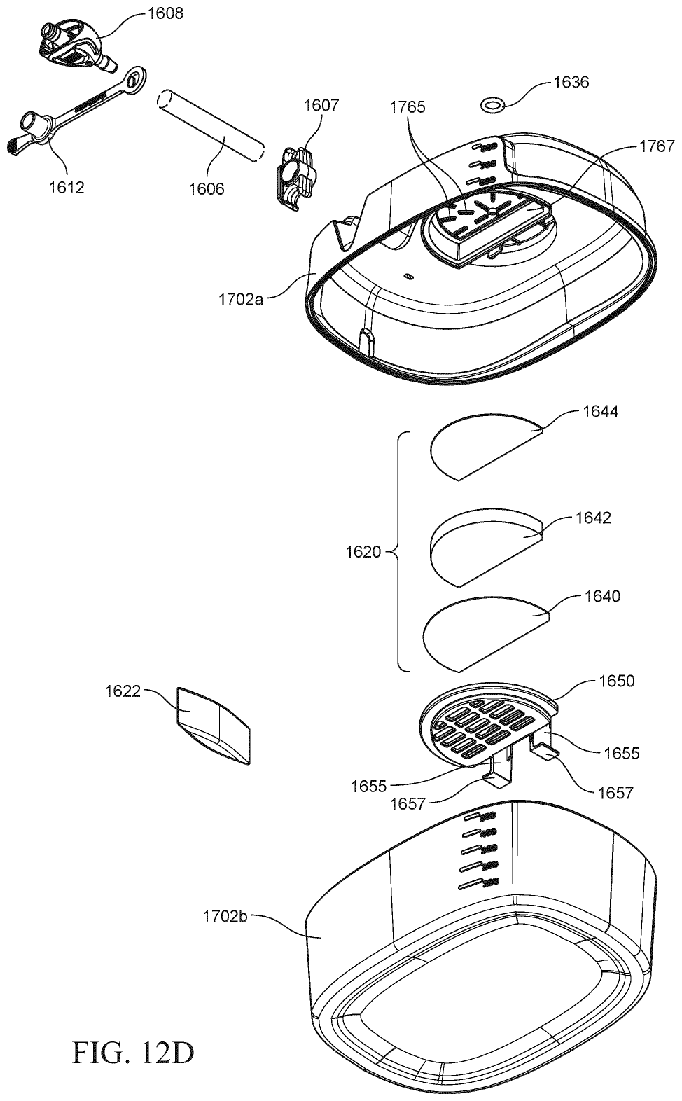

With reference to FIG. 11D, the filter assembly 1620 can be used to filter out air passing from the interior space 1624 of the canister body 1602 to the pump assembly through the opening or passageway 1621 in the connector interface 1618 and can include a hydrophobic filter 1640, an odor filter 1642, and a dust filter 1644 that can be used to inhibit (e.g., prevent) dust or other particulates from passing through to the pump assembly. The odor filter 1642 can also be configured to filter out bacteria from the air flowing through the filter assembly 1620. The hydrophobic filter 1640 can be used to prevent any liquids from escaping from the canister body 1602 and from contacting the odor filter 1642. The odor filter 1642 can include any suitable filter membrane or material, including carbon. For example and without limitation, some arrangements of the odor filter 1642 can include compressed carbon. The filter assembly 1620 is also shown in FIGS. 12C and 12D. The first body portion 1602 can have one or more projections or standoffs formed on an inside surface of the first body portion 1602, similar to or the same as the projections 1765 formed or positioned on the first body portion 1702 of the canister assembly 1700 shown in FIG. 12D. The projections can be used to space the dust filter 1644 away from the planar surface inside the first body portion 1602a, 1702a and allow a greater flow of air through the dust filter 1644.

In some arrangements, the filter 1642 can include a carbon activated foam material. In some arrangements, the filter 1642 can include a compressed carbon element as part of the filtration system in the canister. The carbon element can have various shapes and sizes depending on the canister. Different sizes and shapes for the carbon filter can be employed to increase its effectiveness for different canister shapes. The filter assembly 1620 can optionally be positioned within a recess formed in the first body portion 1602a, such as the recess 1767 formed in the first body portion 1702a, as shown in FIG. 12D.

The filter assembly 1620 can be supported at a lower end or interior end by a base support 1650 that can be configured to provide a support surface for the hydrophobic filter 1640 and/or other components of the filter assembly 1620. The base support 1650 can have one or a plurality of openings 1652 through a main surface 1653 thereof through which air/or other gases can pass as air and/or other gases are being drawn by the pump through the filter assembly 1620.

Some arrangements of the base support 1650 can optionally be configured to support a sensor or sensors and/or other electronics components. With reference to FIG. 12C, some arrangements of the base support 1650 can have a support surface 1654 configured to support a sensor 1658 and/or other electronic components. For example and without limitation, the base support 1650 can have a support surface that is approximately parallel with a top surface of the canister assembly 1600. In some arrangements, the base support 1650 can also have one or more support tabs 1655 (two being shown) to provide additional support for a sensor or sensors and/or other electronics components. For example, the sensor can include a pair of electrodes configured to determine a fill level of the canister or detect that the canister is full responsive to a detection of electric current being conducted between the electrodes via liquid (e.g., wound exudate) aspirated into the canister. The support tabs 1655 can support the pair of electrodes, which can be positioned on the outward facing side of the support tabs 1655.

The support tabs 1655 can extend away from the support surface 1654 toward a bottom of the canister. The support tabs 1655 can have a flange or shield 1657 at a distal end of each of the support tabs 1655 to inhibit liquid (e.g., wound exudate) within the canister from splashing onto the support tabs 1655 and/or the electronics components 1658 (such as, electrodes) and from exposure to the gel packet 1622 or a mound of gelling agent. In some arrangements, the flanges 1657 can each extend at an angle (e.g., at a perpendicular angle) away from the support tabs 1655. In other arrangements, the flanges 1657 can extend at an angle that is greater than or less than 90 degrees relative to the support tabs 1655.

In some arrangements, the electronics components 1658 can optionally be a fill level sensor or a canister full sensor. The fill level sensor can have a wireless transmitter thereon (that can optionally be a near field communication transmitter) that can be configured to communicate status information (such as, detected fill level or whether the canister is full) to a wireless receiver in the pump assembly or otherwise, or can have a wired connection through the canister in communication with the pump assembly.

The base support 1650 can have an annular flange 1660 around a perimeter thereof and a recessed portion 1662 that can be configured to receive and support at least the hydrophobic filter 1640. The base support 1650 can be welded, adhered, or otherwise coupled within an inside surface of the first body portion 1602a of the body 1602 of the canister assembly 1600, optionally, before the first and second portions 1602a, 1602b of the body 1602 are coupled together.

FIGS. 12A-12D illustrate another arrangement of a canister assembly 1700 that can be used with any of the pump assembly arrangements disclosed herein, including any arrangements of the pump assembly 160 disclosed herein. Any arrangements of the canister assembly 1700 disclosed herein can have any of the components, features, or other details of any other canister assembly arrangements disclosed herein, including without limitation any of the arrangements of the canister 162 and/or the canister assembly 1600 described above, in any combination with any of the components, features, or details of the canister assembly 1700 disclosed below. Similarly, any components, features, or other details of any of the other canister or canister assembly arrangements disclosed herein can have any of the components, features, or other details of any arrangements of the canister assembly 1700 disclosed herein in any combination with any of the components, features, or details of the canister or canister assembly.

In any arrangements disclosed herein, the canister assembly 1700 can have a canister body 1702, a conduit or tubing 1606 having a connector 1608 at a distal end thereof, a connector interface 1618, and a filter assembly 1620 housed within the canister body 1700, as described above. The canister body 1700 can have a first or upper portion 1702a and a second or lower portion 1702b and can be any desired size, including 800 mL or approximately 800 mL, or from 600 mL or approximately 600 mL to 1000 mL or approximately 1000 mL. In any arrangements disclosed herein, the canister assembly 1700 can also have a gelling agent 1622 (which can be in a package or bag) positioned within the interior space 1724 of the canister body 1702.

With reference to FIG. 12D, the filter assembly 1620 of the arrangement of the canister assembly 1600, 1700 can include a hydrophobic filter 1640, an odor filter 1642 that can optionally be upstream of the hydrophobic filter 1640, and a dust filter 1644 that can optionally be upstream of the odor filter 1642 and can be used to inhibit (e.g., prevent) dust or other particulates from passing through to the pump assembly. The filter assembly 1620 can be supported at a lower end or interior end by a base support 1650 that can be configured to provide a support surface for the hydrophobic filter 1640 and/or other components of the filter assembly 1620. The base support 1650 can be welded, adhered, or otherwise coupled within an inside surface of the first body portion 1702a of the body 1702 of the canister assembly 1700, optionally, before the first and second portions 1702a, 1702b of the body 1702 are coupled together.

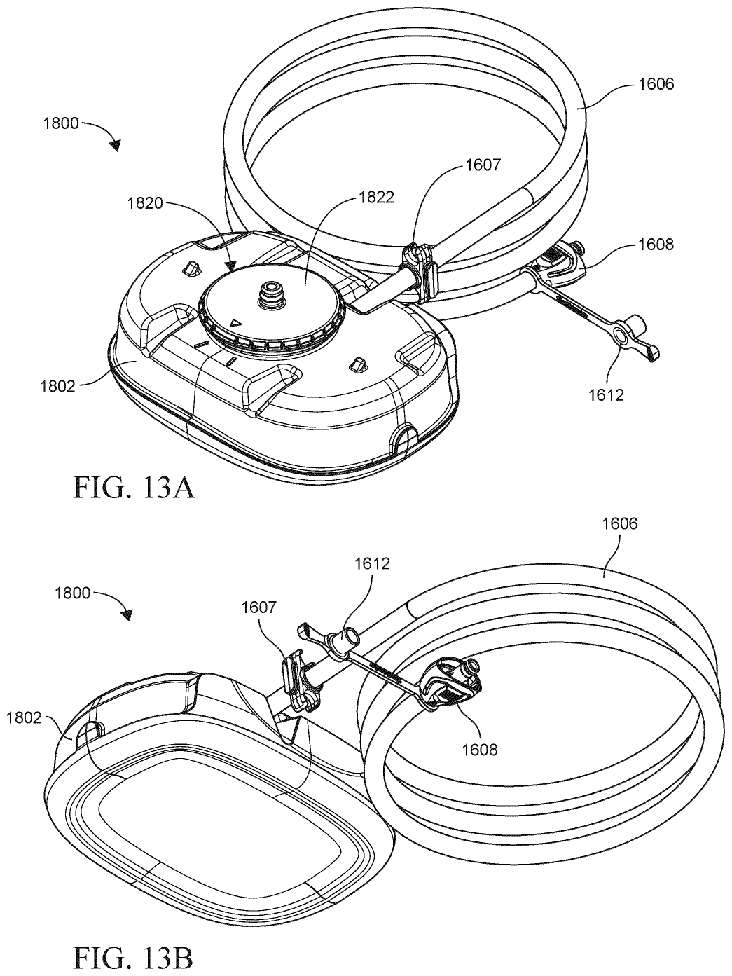

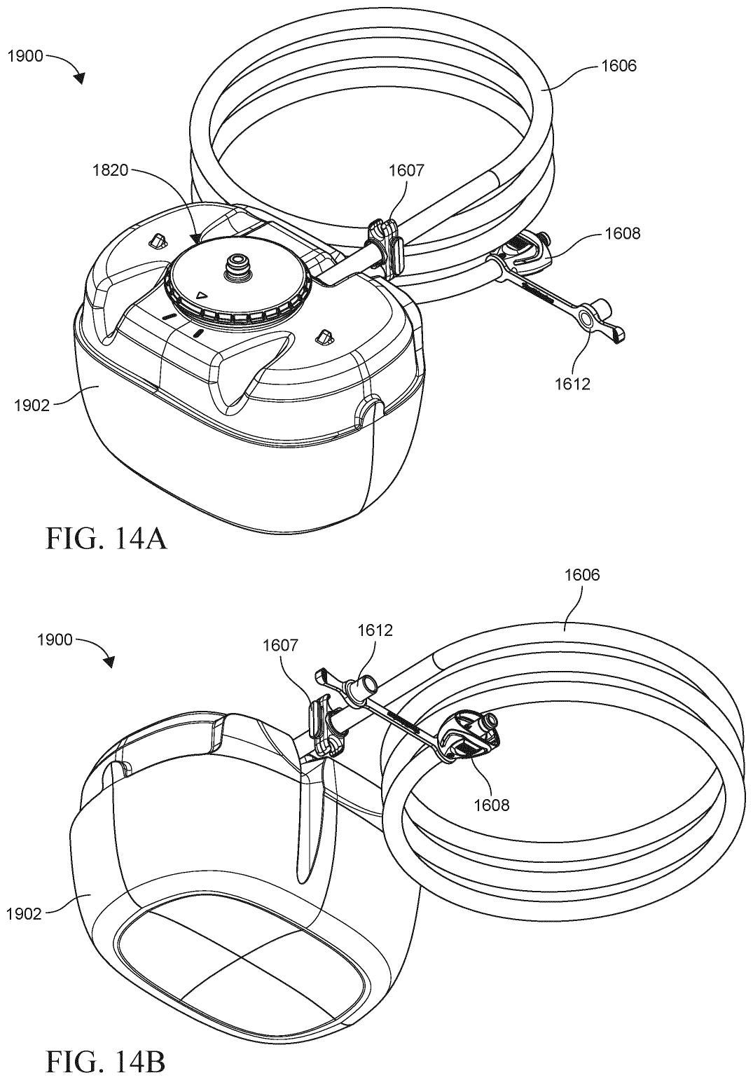

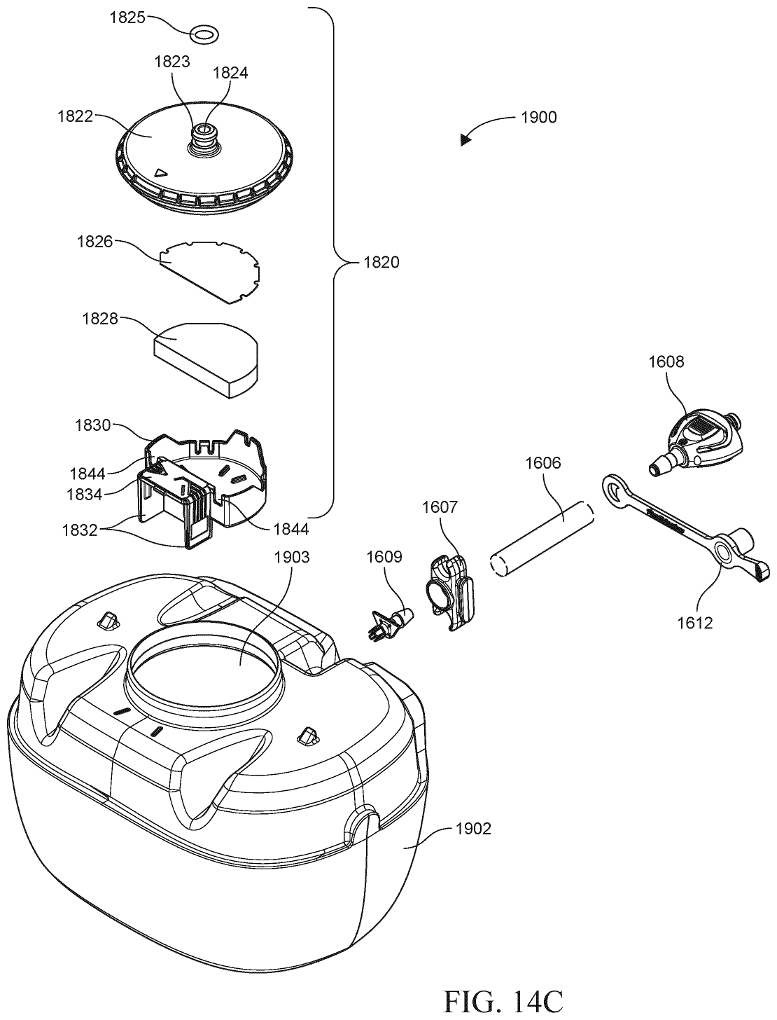

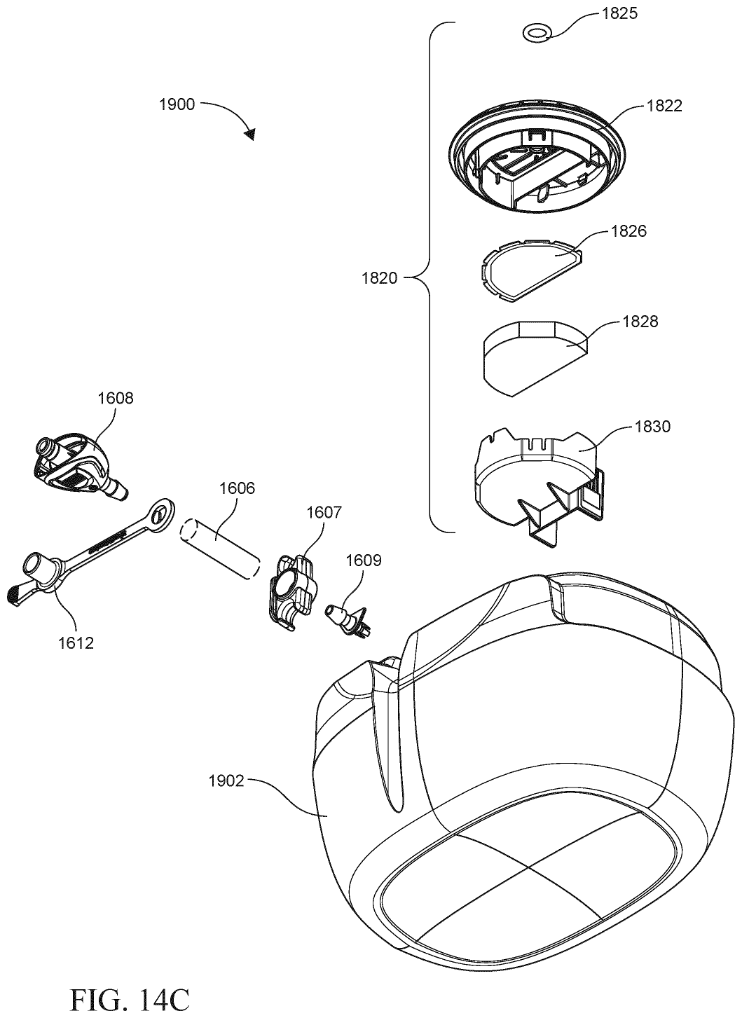

FIGS. 13A-13B illustrate another arrangement of a canister assembly 1800 that can be used with any of the pump assembly arrangements disclosed herein, including any arrangements of the pump assembly 160 disclosed herein. FIGS. 14A-14B illustrate another arrangement of a canister assembly 1900 that can be used with any of the pump assembly arrangements disclosed herein, including any arrangements of the pump assembly 160 disclosed herein.

In any arrangements disclosed herein, any components, features, or other details of the canister assembly 1800 or the canister assembly 1900 can have any of the components, features, or other details of any other canister assembly arrangements disclosed herein, including without limitation any of the arrangements of the canister 182 and canister assemblies 1600, 1700 described above, in any combination with any of the components, features, or details of the canister assembly 1800, 1900 disclosed below. Similarly, any components, features, or other details of any of the other canister or canister assembly arrangements disclosed herein can have any of the components, features, or other details of any arrangements of the canister assembly 1800, 1900 disclosed herein in any combination with any of the components, features, or details of the canister or canister assembly. Some arrangements of the canister assembly 1900 can be the same as the canister assembly 1800 except for the size or volume of the canister assembly. The canister body 1800 can have any desired size or volume, including 300 mL or approximately 300 mL, or from 200 mL or approximately 200 mL to 400 mL or approximately 400 mL. The canister body 1900 can have any desired size or volume, including 800 mL or approximately 800 mL, or from 600 mL or approximately 600 mL to 1000 mL or approximately 1000 mL.

In any arrangements disclosed herein, the canister assembly 1800, 1900 can have a canister body 1802, 1902, respectively, a conduit or tubing 1606 having a clip 1607 and a connector 1608 at a distal end thereof, a connector interface 1818, and a cap assembly 1820 that can be coupled with the canister body 1800. The tubing 1606 can couple with a tubing connector 1609 that can be secured to the canister body 1802, 1902. The canister body 1802 can be blow molded or formed by any desired or suitable method. The canister assembly 1800, 1900 can have a cap assembly 1820 that can have any of the components, features, or other details of any other cap assembly arrangements disclosed herein, including without limitation any of the arrangements of the cap assembly 360 described above, in any combination with any of the components, features, or details of the cap assembly 1820 disclosed below. Similarly, any components, features, or other details of any of the other cap assembly arrangements disclosed herein can have any of the components, features, or other details of any arrangements of the cap assembly 1820 disclosed herein in any combination with any of the components, features, or details of the cap assembly.

In some arrangements, the cap assembly 1820 can be configured to be removably coupled (for example and without limitation, threadedly coupled) with an opening (such as 1903 shown in FIG. 14C) of the canister body 1802, 1902. In some arrangements, the cap assembly 1820 can be welded to the canister body 1802, 1902 or otherwise nonremovably coupled to the canister body. Some arrangements of the cap assembly 1820 can include a cover or first cap member 1822 having a connector interface 1823 that can have an opening 1824 extending axially through a center portion of the first cap member 1822. The connector interface 1823 can project axially away from a first main surface of the first cap member 1822. The connector interface 1823 can have a generally cylindrical shape and an annular flange formed thereon that can be configured to receive a seal, such as an O-ring 1825. The opening 1824 can be configured to provide a fluid passageway for air and/or other gases within the canister body 1802, 1902 to pass and to exit the canister body 1802, 1902 through.

The cap assembly 1820 can include an upper filter 1826 and an odor filter 1828. The upper filter 1826 can be a hydrophobic filter and/or a dust filter. The odor filter 1828 can also be configured to filter out bacteria from the air flowing through the filter 1828. The upper filter 1826 can be used to prevent any liquids from escaping from the canister body 1802, 1902 through the opening 1824 in the first cap member 1822 and can be positioned on either or both sides of the odor filter 1828. The odor filter 1828 can include any suitable filter membrane or material, including carbon. For example and without limitation, some arrangements of the odor filter 1828 can include compressed carbon.

The cap assembly 1820 can also include a base cap support 1830 that can be configured to provide a support surface for one or more of the filters 1826, 1828 and/or other components of the cap assembly 1820. The base cap support 1830 be configured to block or shield the one or more filters 1826, 1828 from exudate and/or other liquids within the canister. In some arrangements, the base cap support 1830 can have a main surface 1840 that can overlap or cover at least a portion of the filter 1828 so as to inhibit or prevent liquid or exudate within the canister 1802, 1902 from splashing onto at least a portion of the odor filter 1828 and/or the upper filter 1826. For example and without limitation, the main surface 1840 can overlap at least 80% of a surface area of a lower main surface of the odor filter 1828, or at least 90% of a surface area of the lower main surface of the filter 1828, or from at least 60% or approximately 60% to 90% or approximately 90% of a surface area of the first main surface of the filter 1828.

The base cap support 1830 can have one or more openings 1844 formed therein that air and/or other gases can pass through as the air and/or other gases are being drawn through the cap assembly 1820 when the pump is in operation. The cap assembly 1820 can be configured such that all air or gas or substantially all air or gas coming from the canister body 1802, 1902 must pass through the filter 1828 before passing through the opening 1844 in the cap assembly 1820. In some arrangements, there can be 3 or more, 4 or more, 5 or more openings 1844 formed in the base cap support 1830. The openings 1844 can be formed in walls that are perpendicular to a top main surface of the canister body 1802, 1902 so that exudate is less likely to splash or otherwise pass through the openings 1844—e.g., the openings 1844 can be formed in vertical walls of the base cap support 1830.

In any arrangements disclosed herein, the canister assembly 1800, 1900 can also have a gelling agent (which can be in a package or bag) positioned within an interior space of the canister body 1802, 1902 configured to increase a thickness or viscosity of the liquid, which can include wound exudate, within the interior space of the canister.

FIG. 15A is a top, front, and left side perspective view of an arrangement of a device 2000 for applying negative pressure to a wound.

FIG. 15B is a front view of the arrangement of the device 2000 of FIG. 15A.

FIG. 15C is a back view of the arrangement of the device 2000 of FIG. 15A.

FIG. 15D is a right side view of the arrangement of the device 2000 of FIG. 15A.

FIG. 15E is a left view of the arrangement of the device 2000 of FIG. 15A.

FIG. 15F is a top view of the arrangement of the device 2000 of FIG. 15A.

FIG. 15G is a bottom view of the arrangement of the device 2000 of FIG. 15A.

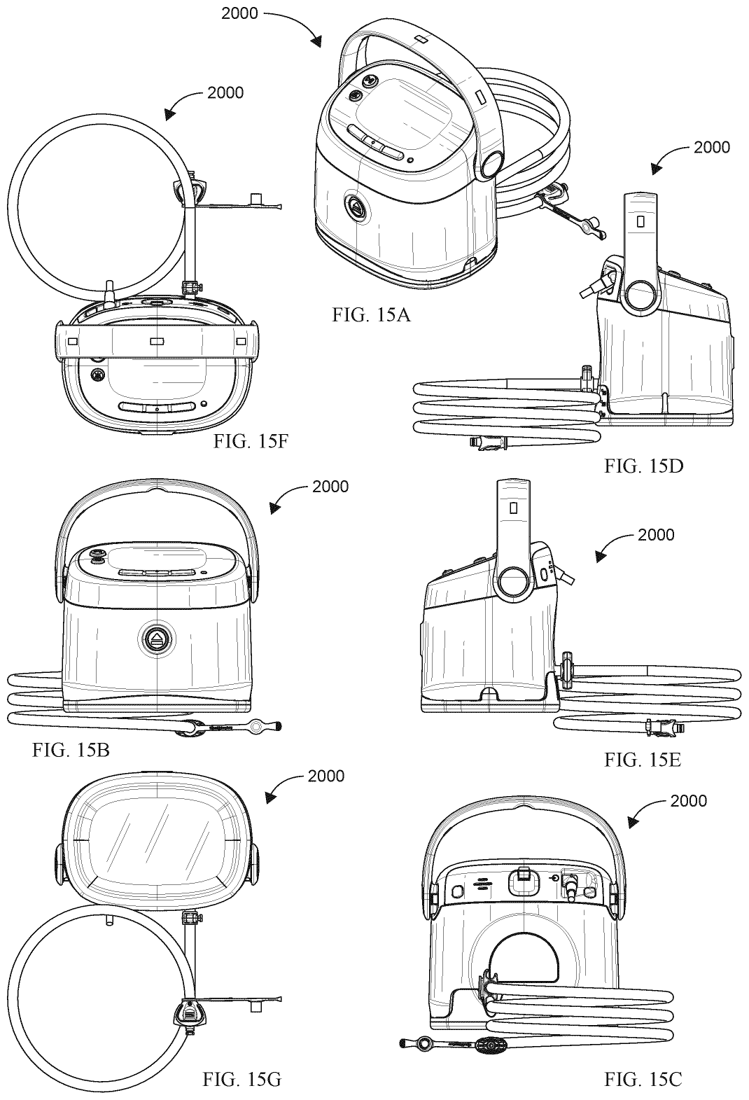

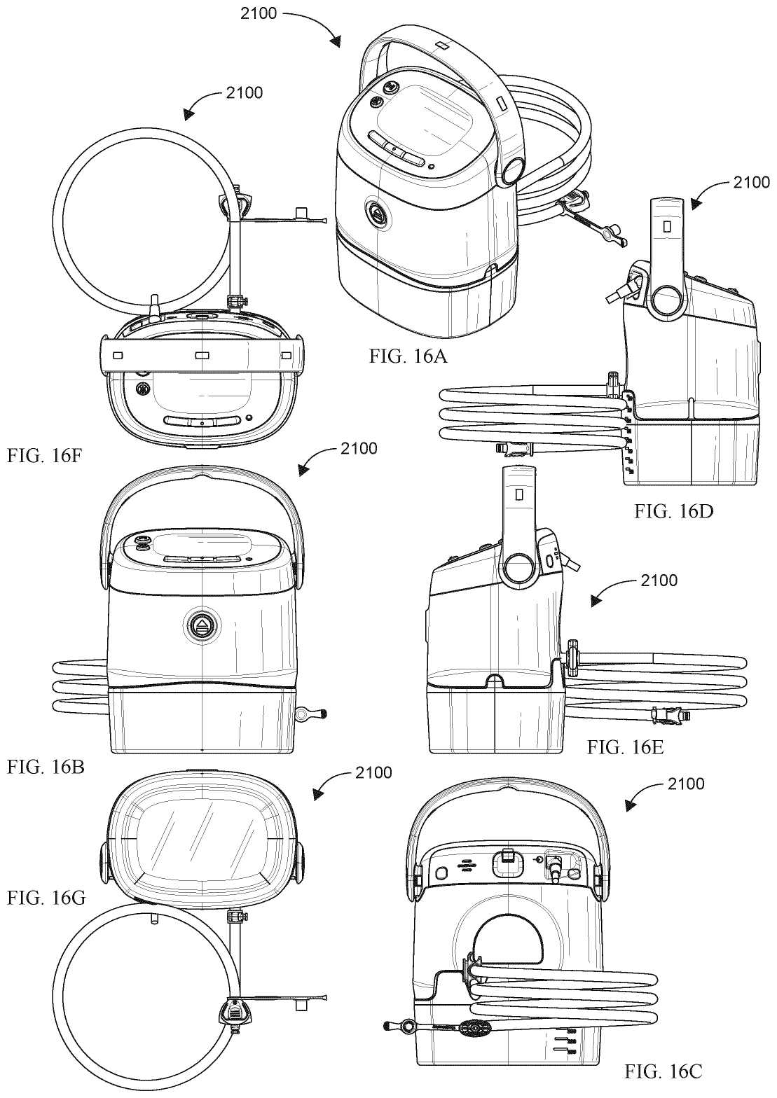

FIG. 16A is a top, front, and left side perspective view of another arrangement of a device 2100 for applying negative pressure to a wound.

FIG. 16B is a front view of the arrangement of the device 2100 of FIG. 16A.

FIG. 16C is a back view of the arrangement of the device 2100 of FIG. 16A.

FIG. 16D is a right side view of the arrangement of the device 2100 of FIG. 16A.

FIG. 16E is a left view of the arrangement of the device 2100 of FIG. 16A.

FIG. 16F is a top view of the arrangement of the device 2100 of FIG. 16A.

FIG. 16G is a bottom view of the arrangement of the device 2100 of FIG. 16A.

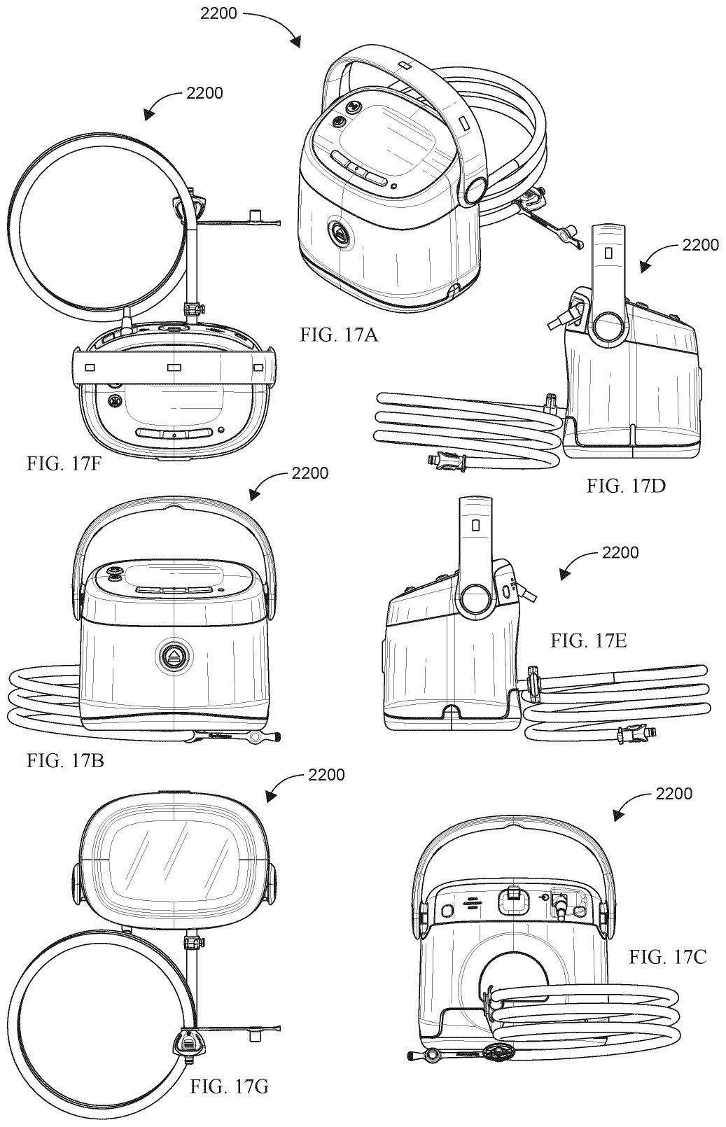

FIG. 17A is a top, front, and left side perspective view of another arrangement of a device 2200 for applying negative pressure to a wound.

FIG. 17B is a front view of the arrangement of the device 2200 of FIG. 17A.

FIG. 17C is a back view of the arrangement of the device 2200 of FIG. 17A.

FIG. 17D is a right side view of the arrangement of the device 2200 of FIG. 17A.

FIG. 17E is a left view of the arrangement of the device 2200 of FIG. 17A.

FIG. 17F is a top view of the arrangement of the device 2200 of FIG. 17A.

FIG. 17G is a bottom view of the arrangement of the device 2200 of FIG. 17A.

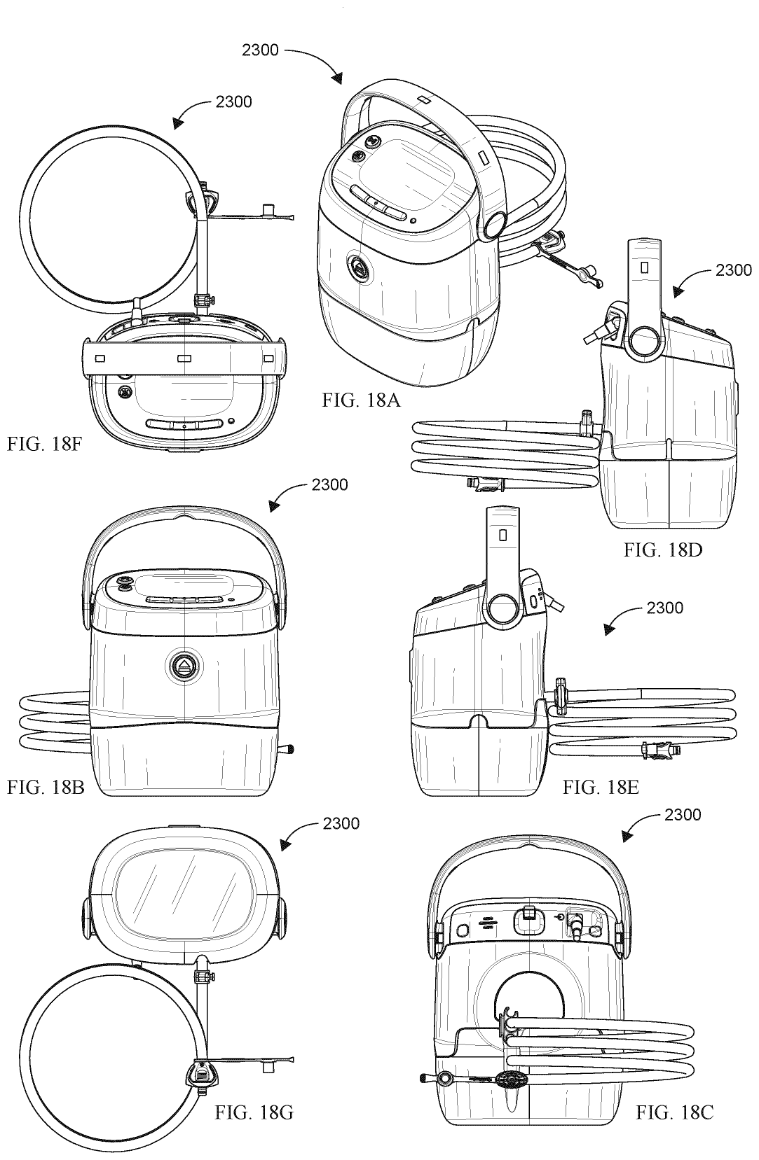

FIG. 18A is a top, front, and left side perspective view of another arrangement of a device 2300 for applying negative pressure to a wound.

FIG. 18B is a front view of the arrangement of the device 2300 of FIG. 18A.

FIG. 18C is a back view of the arrangement of the device 2300 of FIG. 18A.

FIG. 18D is a right side view of the arrangement of the device 2300 of FIG. 18A.

FIG. 18E is a left view of the arrangement of the device 2300 of FIG. 18A.

FIG. 18F is a top view of the arrangement of the device 2300 of FIG. 18A.

FIG. 18G is a bottom view of the arrangement of the device 2300 of FIG. 18A.

In any of the arrangements 2000, 2100, 2200, 2300 shown and described herein, any of the solid lines of such arrangements can be broken lines that are used to denote features that are not part of the claimed ornamental design. The scope of the present disclosure encompasses all lines illustrated, whether broken or solid.

### OTHER VARIATIONS

Although some arrangements describe negative pressure wound therapy, the systems, devices, and/or methods disclosed herein can be applied to other types of therapies usable standalone or in addition to TNP therapy. Systems, devices, and/or methods disclosed herein can be extended to any medical device, and in particular any wound treatment device. For example, systems, devices, and/or methods disclosed herein can be used with devices that provide one or more of ultrasound therapy, oxygen therapy, neurostimulation, microwave therapy, active agents, antibiotics, antimicrobials, or the like. Such devices can in addition provide TNP therapy. The systems and methods disclosed herein are not limited to medical devices and can be utilized by any electronic device.

Any of transmission of data described herein can be performed securely. For example, one or more of encryption, https protocol, secure VPN connection, error checking, confirmation of delivery, or the like can be utilized.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value.

Features, materials, characteristics, or groups described in conjunction with a particular aspect, arrangement, or example are to be understood to be applicable to any other aspect, arrangement or example described herein unless incompatible therewith. All of the features disclosed in this specification (including any accompanying claims, abstract and drawings), and/or all of the steps of any method or process so disclosed, can be combined in any combination, except combinations where at least some of such features and/or steps are mutually exclusive. The protection is not restricted to the details of any foregoing arrangements. The protection extends to any novel one, or any novel combination, of the features disclosed in this specification (including any accompanying claims, abstract and drawings), or to any novel one, or any novel combination, of the steps of any method or process so disclosed.

While certain arrangements have been described, these arrangements have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some arrangements, the actual steps taken in the processes illustrated and/or disclosed may differ from those shown in the figures. Depending on the arrangement, certain of the steps described above may be removed, others may be added. For example, the actual steps and/or order of steps taken in the disclosed processes may differ from those shown in the figure. Depending on the arrangement, certain of the steps described above may be removed, others may be added. For instance, the various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific arrangements disclosed above may be combined in different ways to form additional arrangements, all of which fall within the scope of the present disclosure.

User interface screens illustrated and described herein can include additional and/or alternative components. These components can include menus, lists, buttons, text boxes, labels, radio buttons, scroll bars, sliders, checkboxes, combo boxes, status bars, dialog boxes, windows, and the like. User interface screens can include additional and/or alternative information. Components can be arranged, grouped, displayed in any suitable order.

Conditional language used herein, such as, among others, “can,” “could”, “might,” “may,” “e.g.,” and the like, unless specifically stated otherwise, or otherwise understood within the context as used, is generally intended to convey that certain arrangements include, while other arrangements do not include, certain features, elements and/or states. Thus, such conditional language is not generally intended to imply that features, elements and/or states are in any way required for one or more arrangements or that one or more arrangements necessarily include logic for deciding, with or without author input or prompting, whether these features, elements and/or states are included or are to be performed in any particular arrangement. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain arrangements require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain arrangements, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

Although the present disclosure includes certain arrangements, examples and applications, it will be understood by those skilled in the art that the present disclosure extends beyond the specifically disclosed arrangements to other alternative arrangements and/or uses and obvious modifications and equivalents thereof, including arrangements which do not provide all of the features and advantages set forth herein. Accordingly, the scope of the present disclosure is not intended to be limited by the specific disclosures of preferred arrangements herein, and may be defined by claims as presented herein or as presented in the future.

1. A negative pressure wound therapy device, comprising:
a negative pressure source including an inlet and an outlet, the negative pressure source configured to provide, via a fluid flow path, negative pressure to a wound covered by a wound dressing to aspirate fluid from the wound;
a first noise reduction chamber positioned in the fluid flow path downstream of the negative pressure source and in fluid communication with the outlet of the negative pressure source, the first noise reduction chamber being configured to reduce noise generated as a result of aspirating fluid from the wound and/or reduce a level of pressure pulses in the fluid that is advanced through the negative pressure source, the first noise reduction chamber including an inlet and an outlet;
a second noise reduction chamber positioned in the fluid flow path downstream of the negative pressure source and in fluid communication with the outlet of the first noise reduction chamber, the second noise reduction chamber being configured to reduce noise generated as a result of aspirating fluid from the wound and/or reduce a level of pressure pulses in the fluid that is advanced through the negative pressure source and the first noise reduction chamber, the second noise reduction chamber including an inlet and an outlet;
wherein:
the second noise reduction chamber is spaced apart from the first noise reduction chamber; and
the second noise reduction chamber is different than the first noise reduction chamber.

2. The device of claim 1, wherein the first noise reduction chamber is configured to reduce noise generated by the negative pressure source and/or reduce a level of pressure pulses in the fluid that is advanced through the negative pressure source.

3. The device of claim 1, further comprising a check valve positioned in the fluid flow path and configured to prevent fluid from flowing in a reverse direction back toward the negative pressure source.

4. The device of claim 3, wherein the first noise reduction chamber is configured to reduce a level of pressure pulses in the fluid that is advanced through the negative pressure source to reduce noise generated by the check valve.

5. The device of claim 3, wherein the first noise reduction chamber is positioned upstream of the check valve and the second noise reduction chamber is positioned downstream of the check valve.

6. The device of claim 1, wherein the second noise reduction chamber is positioned in series with the first noise reduction chamber and downstream from the first noise reduction chamber.

7. The device of claim 2, wherein the second noise reduction chamber is more proximal to an exhaust of the device than the first noise reduction chamber.

8. The device of claim 1, further comprising foam positioned in at least one of the first and the second noise reduction chambers.

9. The device of claim 1, wherein the first noise reduction chamber comprises an inner wall extending across a majority of a distance between a first wall and a second wall of the first noise reduction chamber positioned adjacent to or opposite the first wall such that an opening is formed between an end of the inner wall and the second wall, the first noise reduction chamber being configured to create a passageway between the inlet and the outlet of the first noise reduction chamber that requires the fluid passing through the first noise reduction chamber to pass through the opening formed between the end of the inner wall segment and the second wall.

10. The device of claim 1, wherein an internal volume in the first and second noise reduction chambers is greater than a volume within a first conduit in fluid communication with the inlet of at least one of the first or second noise reduction chambers and is greater than a volume within a second conduit in fluid communication with the outlet of at least one of the first or second noise reduction chambers.

11. The device of claim 1, further comprising a flow module including one or more pressure sensors and a solenoid.

12. The device of claim 1, wherein the negative pressure source comprises a motor, and wherein the device further comprises a power source configured to power the motor.

13. The device of claim 1, comprising a canister coupleable with the device and configured to collect fluid aspirated from the wound as a result negative pressure being provided by the negative pressure source to the wound and a cap coupled with an opening on the canister.

14. The device of claim 13, further comprising a filter coupled with or supported by the cap.

15. The device of claim 14, wherein the filter comprises a carbon filter.

16. The device of claim 13, further comprising a hydrophobic filter coupled with or supported by the cap.

17. A negative pressure wound therapy system comprising:
a pump device comprising a source of negative pressure configured to be fluidically connected to a wound covered by a wound dressing;
a canister coupleable with the pump device and configured to collect fluid aspirated from a wound as a result of negative pressure being provided to the wound by the source of negative pressure; and
a canister release mechanism coupled with the pump device and comprising an actuator coupled with one or more movable latches, the canister release mechanism being configured to cause the pump device to disengage the canister from the pump device when the actuator is depressed;
wherein:
the one or more latches are configured to move between a first position in which the one or more latches secure the canister to the pump device and a second position in which the one or more latches release the canister from the pump device when the actuator is depressed.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A negative pressure wound therapy system comprising:
a device comprising a negative pressure pump actuated by a pump motor, a battery, a display, a lower core assembly, and an upper support within a housing;
a canister coupleable with the device and configured to collect fluid aspirated from a wound as a result negative pressure being provided by the negative pressure pump to a wound covered by a wound dressing; and
a cap coupled with an opening on the canister;
wherein:
the lower core assembly is configured to receive and support at least the pump motor and the battery;
the upper support is coupled with the lower core assembly, the upper support extending above the lower core assembly;
the upper support is configured to receive and support at least the display of the pump assembly; and
the display can be removed from the pump assembly by removing the housing and by removing the upper support from the pump assembly.

33. (canceled)

34. (canceled)

35. (canceled)

36. (canceled)

37. (canceled)

38. (canceled)

39. (canceled)

40. (canceled)

41. (canceled)
